(19) United States
(12) Patent Application Publication
(10) Pub. No.: US 2025/0049923 A1
(43) Pub. Date: Feb. 13, 2025

(54) NOVEL COMPOSITION

(71) Applicant: Arecor Limited, Saffron Walden (GB)

(72) Inventors: Jan JEZEK, Saffron Walden (GB); Adrian PODMORE, Saffron Walden (GB); Swapneel DESHPANDE, Saffron Walden (GB); Alexis HENNESSY, Saffron Walden (GB)

(21) Appl. No.: 18/720,018

(22) PCT Filed: Dec. 15, 2022
(86) PCT No.: PCT/GB2022/053256

§ 371 Date: Jun. 14, 2024

(30) Foreign Application Priority Data
Dec. 15, 2021 (GB) 2118175.5

(51) Int. Cl. A61K 47/10; A61K 9/00; A61K 9/08; A61K 31/4184

(52) U.S. Cl. CPC A61K 47/10; A61K 9/0019; A61K 9/08; A61K 31/4184

(57) ABSTRACT

There is provided inter alia a storage stable liquid composition concentrate comprising bendamustine or a pharmaceutically acceptable salt thereof and a liquid solvent in which said bendamustine or a pharmaceutically acceptable salt thereof is dissolved;
wherein said composition comprises:
(i) a mixture of two or more different polyethylene glycol (PEG) polymers, wherein each polyethylene glycol has an average molecular weight in the range 150-650 Da; and
(ii) 5-20% (v/v) of water.

This invention relates to storage stable liquid compositions of bendamustine, in particular to ready-to-dilute liquid composition concentrates of bendamustine with low viscosity.

### BACKGROUND

Bendamustine is an alkylating drug used as a first line of treatment in chronic lymphocytic leukaemia (CLL), multiple myeloma and non-Hodgkin lymphoma (NHL), as a sole therapy or in combination with other agents including etoposide, rituximab, fludarabine, mitoxantrone, methotrexate, prednisone, vincristine or ⁹⁰Y-ibritumomab tiuxetan.

Bendamustine (4-[5-[bis(2-chloroethyl)amino]-1-methylbenzimidazol-2-yl]butanoic acid) is a nitrogen mustard-derivative with a molecular weight of 359 Da. Bendamustine may also be used in the form of its hydrochloride salt, with molecular weight of 395 Da. It is a strong alkylating agent, causing intra- and inter-strand cross-links between DNA and RNA bases of mutagenic cells, resulting in strong cytotoxic properties. Bendamustine is rapidly hydrolysed to mono-hydroxy-bendamustine and dihydroxy-bendamustine, which have little or no activity (Darwish et al., Cancer Chemother Pharmacol (2015) 75:1143-1154).

Both lyophilized and liquid bendamustine products have been approved for use in humans in the US. One of the liquid products has been discontinued due to safety issues. The summary of the products (the compositions and instructions for storage and use) is as follows:

Treanda for Injection® was approved in 2008 and is marketed as a lyophilized powder requiring reconstitution and subsequent dilution before use before use. It is supplied in two formats, both to be stored at room temperature (15-30° C. or 59-86° F.). The first is a lyophilized powder containing 25 mg bendamustine hydrochloride and 42.5 mg mannitol, which is reconstituted with 5 mL WFI (Water for Injection) USP. The second is a lyophilized powder containing 100 mg bendamustine hydrochloride and 170 mg mannitol, which is reconstituted with 20 mL WFI USP. The pH of the reconstituted solution is 2.5-3.5. The dose needed (dependent on condition and patient body weight) is then to be withdrawn within 30 min of reconstitution, put in a 500 mL infusion bag and diluted to a final concentration of bendamustine hydrochloride of 5 mg/mL in 0.9% sodium chloride or 2.5% dextrose/0.45% sodium chloride. This admixture is stable for 24 h at 2-8° C. or 3 hours at room temperature (15-30° C. or 59-86° F.) and should be injected within this period.

Treanda Injection® was approved in 2008 but discontinued in 2015, and was marketed as a colourless to yellow liquid ready-to-dilute format, supplied in two different forms. The first is 0.5 mL of liquid in a vial containing 45 mg of bendamustine hydrochloride, 162 mg of propylene glycol and 293 mg of N,N-dimethylacetamide. The second is 2 mL of liquid in a vial containing 180 mg of bendamustine hydrochloride, 648 mg of propylene glycol and 1172 mg of N,N-dimethylacetamide. The unopened vials are stable for up to 2 years at 2-8° C. The dose needed (dependent on condition and patient body weight) is withdrawn from a vial, put in a 500 mL infusion bag and diluted to a final concentration of bendamustine of 5 mg/mL in 0.9% sodium chloride or 2.5% dextrose/0.45% sodium chloride. This product was discontinued due to interactions between the N,N-dimethylacetamide solvent and closed-system transfer devices, adapters and syringes containing polycarbonate or acrylonitrile-butadiene-styrene.

Bendeka® and Belrapzo® were approved in 2016 and are supplied as a multiple-dose vial 4 mL of a ready-to-dilute liquid containing 100 mg (25 mg/mL) bendamustine hydrochloride, 20 mg (5 mg/mL) monothioglycerol, 0.4 mL propylene glycol and 3.6 mL PEG400. The unopened vial is stable for up to 2 years at 2-8° C. The dose needed (dependent on condition and patient body weight) is withdrawn from the vial, put in a 50 mL infusion bag and diluted to a final volume of 50 mL in 0.9% sodium chloride or 2.5% dextrose/0.45% sodium chloride or 5% dextrose. This admixture is stable for 24 h at 2-8° C. or 3 hours at room temperature (15-30° C. or 59-86° F.) and should be injected within this period. However, both liquid products have relatively high viscosity (94.2 mPa·s, measured using the Viscosity Assessment Method described in the present Examples) which may negatively impact dosing accuracy and handling.

As such, there is a need for liquid ready-to-dilute composition concentrates of bendamustine with a good safety profile and sufficient stability in liquid form, with relatively low viscosity.

WO2011/094565A1 (Eagle Pharmaceuticals, Inc.) discloses non-aqueous compositions containing bendamustine and a pharmaceutically acceptable fluid which can be PEG, propylene glycol or a mixture thereof.

US2020/0297849A1 (Good Health, LLC) discloses liquid pharmaceutical compositions comprising bendamustine, at least one cyclodextrin, at least one non-aqueous solvent, at least about 2% water v/v and at least one antioxidant.

WO2020/035806A1 (Hospira Australia Pty Ltd) discloses concentrated liquid pharmaceutical compositions comprising bendamustine, a non-aqueous solvent, and at least about 2% water.

### SUMMARY OF THE INVENTION

The present invention provides, inter alia, a storage stable liquid composition concentrate comprising bendamustine or a pharmaceutically acceptable salt thereof and a liquid solvent in which said bendamustine or a pharmaceutically acceptable salt thereof is dissolved; wherein said composition comprises:
(i) a mixture of two or more different polyethylene glycol (PEG) polymers, wherein each polyethylene glycol has an average molecular weight in the range 150-650 Da; and
(ii) 5-20% (v/v) of water.

### DETAILED DESCRIPTION OF THE INVENTION

As used herein, a liquid ready-to-dilute composition concentrate (also referred to herein as a “composition concentrate of the invention”) is a liquid composition intended for long-term storage in a suitable container (e.g. a sealed vial) in which the concentration of bendamustine is higher than that required for administration (typically intravenous administration). The ready-to-dilute liquid composition concentrate is therefore diluted to the required concentration shortly before the administration (typically intravenous administration).

The ready-to-dilute composition concentrates of bendamustine described herein comprising 5-20% (v/v) water and a mixture of PEG polymers have excellent storage stability and lower viscosity than the currently marketed ready-to-dilute bendamustine composition Bendeka®. Given that bendamustine is prone to rapid hydrolysis, the stability of formulations of the invention comprising 5-20% water (v/v) demonstrated in the present Examples is surprising.

The term “v/v” means “volume per volume” and is used to express the concentration of a liquid substance in the composition on a volume per volume basis. For example, when the liquid solvent comprises 10% (v/v) of water, this means that there is about 10 mL of water in every 100 mL of the composition concentrate.

The dalton (abbreviation Da) will be known to the skilled person as a widely used unit of atomic or molecular mass and is an alternative name for the unified atomic mass unit (symbol “u”). One dalton corresponds to one twelfth of the mass of an unbound neutral atom of carbon-12 in its nuclear and electronic ground state and at rest.

In the composition concentrates of the invention bendamustine may be present in the form of a pharmaceutically acceptable salt. Examples of pharmaceutically acceptable salts include hydrochloride, hydrobromide, citrate, formate, acetate, tartrate, sulphate, tosylate, nitrate, mesylate, napsylate, besylate, oxalate, maleate, phosphate, pamoate, fumarate, hippurate, succinate and carbonate. In one embodiment, the bendamustine form is bendamustine hydrochloride. For the avoidance of doubt, any reference herein to “bendamustine” is also intended to cover a pharmaceutically acceptable salt of bendamustine, in particular the hydrochloride salt, unless stated otherwise.

In one embodiment, the concentration of bendamustine or pharmaceutically acceptable salt thereof in the composition concentrate is 25-250 mM, e.g. 25-225 mM, 25-200 mM, 25-175 mM, 25-150 mM, 25-125 mM, 25-100 mM, 25-75 mM, 35-250 mM, 35-225 mM, 35-200 mM, 35-175 mM, 35-150 mM, 35-125 mM, 35-100 mM, 35-75 mM, 45-250 mM, 45-225 mM, 45-200 mM, 45-175 mM, 45-150 mM, 45-125 mM, 45-100 mM, or 45-75 mM, such as about 61 mM.

As described in detail in Example 4, the present inventors observed that starting with the marketed bendamustine composition Bendeka® which is non-aqueous and contains a mixture of propylene glycol (1,2-propanediol) and PEG400 as liquid solvent, replacing a proportion of the liquid solvent of Bendeka® with water (10%) resulted in a decrease in stability. However, if the propylene glycol/PEG400 mixture was replaced with a mixture of two PEG polymers of different molecular weight, then the destabilizing effect of adding water was surprisingly reduced.

As shown in Example 5, for composition concentrates containing bendamustine and a mixture of PEG compounds as liquid solvent, as the proportion of water as solvent in the composition concentrate is increased and the relative proportion of the PEG mixture decreased, the greater the rate of formation of impurities. The present inventors have found that having 5-20% (v/v) water in the composition concentrate provides an optimum balance of i) storage stability; ii) viscosity; and iii) solubility for excipients. In one embodiment, the composition concentrate comprises 5-19% (v/v), 5-18% (v/v), 5-17% (v/v), 5-16% (v/v), 5-15% (v/v), 5-14% (v/v), 5-13% (v/v), 5-12% (v/v), 5-11% (v/v), 5-10% (v/v), 6-20% (v/v), 6-19% (v/v), 6-18% (v/v), 6-17% (v/v), 6-16% (v/v), 6-15% (v/v), 6-14% (v/v), 6-13% (v/v), 6-12% (v/v), 6-11% (v/v), 6-10% (v/v), 7-20% (v/v), 7-19% (v/v), 7-18% (v/v), 7-17% (v/v), 7-16% (v/v), 7-15% (v/v), 7-14% (v/v), 7-13% (v/v), 7-12% (v/v), 7-11% (v/v), 7-10% (v/v), 8-20% (v/v), 8-19% (v/v), 8-18% (v/v), 8-17% (v/v), 8-16% (v/v), 8-15% (v/v), 8-14% (v/v), 8-13% (v/v), 8-12% (v/v), 8-11% (v/v), 8-10% (v/v), 9-20% (v/v), 9-19% (v/v), 9-18% (v/v), 9-17% (v/v), 9-16% (v/v), 9-15% (v/v), 9-14% (v/v), 9-13% (v/v), 9-12% (v/v), 9-11% (v/v) or 9-10% (v/v) of water, such as about 10% (v/v). Suitably the water is water for injection (WFI).

As shown in Example 6, the viscosity of composition concentrates of the invention was evaluated and was found to be considerably lower that the formulation used for Bendeka® (currently marketed bendamustine formulation). Thus, composition concentrates of the invention are expected to have comparable or improved storage stability, and reduced viscosity, compared to Bendeka®. Based on the teaching in WO2020/035806A1 (Hospira Australia Pty Ltd) that a small molecule organic solvent such as propylene glycol should be included in the composition to aid viscosity reduction, this finding was surprising. Thus, in one embodiment, the viscosity of the composition concentrate is lower than that of the commercially available formulation of Bendeka® (Formulation 7 of Example 4) e.g. 5% lower, 10% lower, 20% lower or 30% lower. In one embodiment, the viscosity of the composition concentrate at 21° C. is below 90 mPa·s, such as below 80 mPa·s or below 70 mPa·s, suitably as determined using the Viscosity Assessment Method described in General Methods, using capillary extrusion rheometer (mVROC).

Suitably the composition concentrate of the invention comprises two (rather than three, four etc.) different polyethylene glycol (PEG) polymers, wherein each polyethylene glycol has an average molecular weight in the range 150-650 Da.

In a preferred embodiment at least one of the different polyethylene glycol (PEG) polymers is PEG300. In one embodiment, in the composition concentrate of the invention the two different polyethylene glycol (PEG) polymers are selected from the group consisting of PEG200, PEG300, PEG400 and PEG600; and are suitably selected from the group consisting of PEG200, PEG300 and PEG400. In a preferred embodiment, the two different polyethylene glycol polymers are PEG300 and PEG400.

In one embodiment, in the mixture of two different polyethylene glycol (PEG) polymers, the polyethylene glycol polymer with lower average molecular weight is present in an equal or a greater proportion, especially a greater proportion, than the other polyethylene glycol polymer (% v/v). In one embodiment, the ratio of lower average molecular weight PEG to higher average molecular weight PEG is between 1.5:1 and 10:1 (% v/v), e.g. between 3:1 and 8:1, such as about 7:1. In one embodiment, the mixture of two different polyethylene glycol (PEG) polymers is present in the composition in a total amount of 80-95% (v/v), such as 81-95% (v/v), 82-95% (v/v), 83-95% (v/v), 84-95% (v/v), 85-95% (v/v), 86-95% (v/v), 87-95% (v/v), 88-95% (v/v), 89-95% (v/v), 90-95% (v/v), 80-94% (v/v), 81-94% (v/v), 82-94% (v/v), 83-94% (v/v), 84-94% (v/v), 85-94% (v/v), 86-94% (v/v), 87-94% (v/v), 88-94% (v/v), 89-94% (v/v), 90-94% (v/v), 80-93% (v/v), 81-93% (v/v), 82-93% (v/v), 83-93% (v/v), 84-93% (v/v), 85-93% (v/v), 86-93% (v/v), 87-93% (v/v), 88-93% (v/v), 89-93% (v/v), 90-93% (v/v), 80-92% (v/v), 81-92% (v/v), 82-92% (v/v), 83-92% (v/v), 84-92% (v/v), 85-92% (v/v), 86-92% (v/v), 87-92% (v/v), 88-92% (v/v), 89-92% (v/v), 90-92% (v/v), 80-91% (v/v), 81-91% (v/v), 82-91% (v/v), 83-91% (v/v), 84-91% (v/v), 85-91% (v/v), 86-91% (v/v), 87-91% (v/v), 88-91% (v/v), 89-91% (v/v), 90-91% (v/v), 80-90% (v/v), 81-90% (v/v), 82-90% (v/v), 83-90% (v/v), 84-90% (v/v), 85-90% (v/v), 86-90% (v/v), 87-90% (v/v), 88-90% (v/v), 89-90% (v/v) or about 90% (v/v).

In one embodiment, the composition concentrate comprises the mixture of two different polyethylene glycol (PEG) polymers as defined herein in at total amount of 80-95% (v/v), and 5-20% (v/v) water. In one embodiment, the composition concentrate comprises the mixture of two different polyethylene glycol (PEG) polymers as defined herein in at total amount of 81-95% (v/v), and 5-19% (v/v) water. In one embodiment, the composition concentrate comprises the mixture of two different polyethylene glycol (PEG) polymers as defined herein in at total amount of 82-95% (v/v), and 5-18% (v/v) water. In one embodiment, the composition concentrate comprises the mixture of two different polyethylene glycol (PEG) polymers as defined herein in at total amount of 83-95% (v/v), and 5-17% (v/v) water. In one embodiment, the composition concentrate comprises the mixture of two different polyethylene glycol (PEG) polymers as defined herein in at total amount of 84-95% (v/v), and 5-16% (v/v) water. In one embodiment, the composition concentrate comprises the mixture of two different polyethylene glycol (PEG) polymers as defined herein in at total amount of 85-95% (v/v), and 5-15% (v/v) water. In one embodiment, the composition concentrate comprises the mixture of two different polyethylene glycol (PEG) polymers as defined herein in at total amount of 86-95% (v/v), and 5-14% (v/v) water. In one embodiment, the composition concentrate comprises the mixture of two different polyethylene glycol (PEG) polymers as defined herein in at total amount of 87-95% (v/v), and 5-13% (v/v) water. In one embodiment, the composition concentrate comprises the mixture of two different polyethylene glycol (PEG) polymers as defined herein in at total amount of 88-95% (v/v), and 5-12% (v/v) water. In one embodiment, the composition concentrate comprises the mixture of two different polyethylene glycol (PEG) polymers as defined herein in at total amount of 89-95% (v/v), and 5-11% (v/v) water. In one embodiment, the composition concentrate comprises the mixture of two different polyethylene glycol (PEG) polymers as defined herein in at total amount of 90-95% (v/v), and 5-10% (v/v) water. In one embodiment, the composition concentrate comprises the mixture of two different polyethylene glycol (PEG) polymers as defined herein in at total amount of 80-94% (v/v), and 6-20% (v/v) water. In one embodiment, the composition concentrate comprises the mixture of two different polyethylene glycol (PEG) polymers as defined herein in at total amount of 81-94% (v/v), and 6-19% (v/v) water. In one embodiment, the composition concentrate comprises the mixture of two different polyethylene glycol (PEG) polymers as defined herein in at total amount of 82-94% (v/v), and 6-18% (v/v) water. In one embodiment, the composition concentrate comprises the mixture of two different polyethylene glycol (PEG) polymers as defined herein in at total amount of 83-94% (v/v), and 6-17% (v/v) water. In one embodiment, the composition concentrate comprises the mixture of two different polyethylene glycol (PEG) polymers as defined herein in at total amount of 84-94% (v/v), and 6-16% (v/v) water. In one embodiment, the composition concentrate comprises the mixture of two different polyethylene glycol (PEG) polymers as defined herein in at total amount of 85-94% (v/v), and 6-15% (v/v) water. In one embodiment, the composition concentrate comprises the mixture of two different polyethylene glycol (PEG) polymers as defined herein in at total amount of 86-94% (v/v), and 6-14% (v/v) water. In one embodiment, the composition concentrate comprises the mixture of two different polyethylene glycol (PEG) polymers as defined herein in at total amount of 87-94% (v/v), and 6-13% (v/v) water. In one embodiment, the composition concentrate comprises the mixture of two different polyethylene glycol (PEG) polymers as defined herein in at total amount of 88-94% (v/v), and 6-12% (v/v) water. In one embodiment, the composition concentrate comprises the mixture of two different polyethylene glycol (PEG) polymers as defined herein in at total amount of 89-94% (v/v), and 6-11% (v/v) water. In one embodiment, the composition concentrate comprises the mixture of two different polyethylene glycol (PEG) polymers as defined herein in at total amount of 90-94% (v/v), and 6-10% (v/v) water. In one embodiment, the composition concentrate comprises the mixture of two different polyethylene glycol (PEG) polymers as defined herein in at total amount of 80-93% (v/v), and 7-20% (v/v) water. In one embodiment, the composition concentrate comprises the mixture of two different polyethylene glycol (PEG) polymers as defined herein in at total amount of 81-93% (v/v), and 7-19% (v/v) water. In one embodiment, the composition concentrate comprises the mixture of two different polyethylene glycol (PEG) polymers as defined herein in at total amount of 82-93% (v/v), and 7-18% (v/v) water. In one embodiment, the composition concentrate comprises the mixture of two different polyethylene glycol (PEG) polymers as defined herein in at total amount of 83-93% (v/v), and 7-17% (v/v) water. In one embodiment, the composition concentrate comprises the mixture of two different polyethylene glycol (PEG) polymers as defined herein in at total amount of 84-93% (v/v), and 7-16% (v/v) water. In one embodiment, the composition concentrate comprises the mixture of two different polyethylene glycol (PEG) polymers as defined herein in at total amount of 85-93% (v/v), and 7-15% (v/v) water. In one embodiment, the composition concentrate comprises the mixture of two different polyethylene glycol (PEG) polymers as defined herein in at total amount of 86-93% (v/v), and 7-14% (v/v) water. In one embodiment, the composition concentrate comprises the mixture of two different polyethylene glycol (PEG) polymers as defined herein in at total amount of 87-93% (v/v), and 7-13% (v/v) water. In one embodiment, the composition concentrate comprises the mixture of two different polyethylene glycol (PEG) polymers as defined herein in at total amount of 88-93% (v/v), and 7-12% (v/v) water. In one embodiment, the composition concentrate comprises the mixture of two different polyethylene glycol (PEG) polymers as defined herein in at total amount of 89-93% (v/v), and 7-11% (v/v) water. In one embodiment, the composition concentrate comprises the mixture of two different polyethylene glycol (PEG) polymers as defined herein in at total amount of 90-93% (v/v), and 7-10% (v/v) water. In one embodiment, the composition concentrate comprises the mixture of two different polyethylene glycol (PEG) polymers as defined herein in at total amount of 80-92% (v/v), and 8-20% (v/v) water. In one embodiment, the composition concentrate comprises the mixture of two different polyethylene glycol (PEG) polymers as defined herein in at total amount of 81-92% (v/v), and 8-19% (v/v) water. In one embodiment, the composition concentrate comprises the mixture of two different polyethylene glycol (PEG) polymers as defined herein in at total amount of 82-92% (v/v), and 8-18% (v/v) water. In one embodiment, the composition concentrate comprises the mixture of two different polyethylene glycol (PEG) polymers as defined herein in at total amount of 83-92% (v/v), and 8-17% (v/v) water. In one embodiment, the composition concentrate comprises the mixture of two different polyethylene glycol (PEG) polymers as defined herein in at total amount of 84-92% (v/v), and 8-16% (v/v) water. In one embodiment, the composition concentrate comprises the mixture of two different polyethylene glycol (PEG) polymers as defined herein in at total amount of 85-92% (v/v), and 8-15% (v/v) water. In one embodiment, the composition concentrate comprises the mixture of two different polyethylene glycol (PEG) polymers as defined herein in at total amount of 86-92% (v/v), and 8-14% (v/v) water. In one embodiment, the composition concentrate comprises the mixture of two different polyethylene glycol (PEG) polymers as defined herein in at total amount of 87-92% (v/v), and 8-13% (v/v) water. In one embodiment, the composition concentrate comprises the mixture of two different polyethylene glycol (PEG) polymers as defined herein in at total amount of 88-92% (v/v), and 8-12% (v/v) water. In one embodiment, the composition concentrate comprises the mixture of two different polyethylene glycol (PEG) polymers as defined herein in at total amount of 89-92% (v/v), and 8-11% (v/v) water. In one embodiment, the composition concentrate comprises the mixture of two different polyethylene glycol (PEG) polymers as defined herein in at total amount of 90-92% (v/v), and 8-10% (v/v) water. In one embodiment, the composition concentrate comprises the mixture of two different polyethylene glycol (PEG) polymers as defined herein in at total amount of 80-91% (v/v), and 9-20% (v/v) water. In one embodiment, the composition concentrate comprises the mixture of two different polyethylene glycol (PEG) polymers as defined herein in at total amount of 81-91% (v/v), and 9-19% (v/v) water. In one embodiment, the composition concentrate comprises the mixture of two different polyethylene glycol (PEG) polymers as defined herein in at total amount of 82-91% (v/v), and 9-18% (v/v) water. In one embodiment, the composition concentrate comprises the mixture of two different polyethylene glycol (PEG) polymers as defined herein in at total amount of 83-91% (v/v), and 9-17% (v/v) water. In one embodiment, the composition concentrate comprises the mixture of two different polyethylene glycol (PEG) polymers as defined herein in at total amount of 84-91% (v/v), and 9-16% (v/v) water. In one embodiment, the composition concentrate comprises the mixture of two different polyethylene glycol (PEG) polymers as defined herein in at total amount of 85-91% (v/v), and 9-15% (v/v) water. In one embodiment, the composition concentrate comprises the mixture of two different polyethylene glycol (PEG) polymers as defined herein in at total amount of 86-91% (v/v), and 9-14% (v/v) water. In one embodiment, the composition concentrate comprises the mixture of two different polyethylene glycol (PEG) polymers as defined herein in at total amount of 87-91% (v/v), and 9-13% (v/v) water. In one embodiment, the composition concentrate comprises the mixture of two different polyethylene glycol (PEG) polymers as defined herein in at total amount of 88-91% (v/v), and 9-12% (v/v) water. In one embodiment, the composition concentrate comprises the mixture of two different polyethylene glycol (PEG) polymers as defined herein in at total amount of 89-91% (v/v), and 9-11% (v/v) water. In one embodiment, the composition concentrate comprises the mixture of two different polyethylene glycol (PEG) polymers as defined herein in at total amount of 90-91% (v/v), and 9-10% (v/v) water. In one embodiment, the composition concentrate comprises the mixture of two different polyethylene glycol (PEG) polymers as defined herein in at total amount of 80-90% (v/v), and 10-20% (v/v) water. In one embodiment, the composition concentrate comprises the mixture of two different polyethylene glycol (PEG) polymers as defined herein in at total amount of 81-90% (v/v), and 10-19% (v/v) water. In one embodiment, the composition concentrate comprises the mixture of two different polyethylene glycol (PEG) polymers as defined herein in at total amount of 82-90% (v/v), and 10-18% (v/v) water. In one embodiment, the composition concentrate comprises the mixture of two different polyethylene glycol (PEG) polymers as defined herein in at total amount of 83-90% (v/v), and 10-17% (v/v) water. In one embodiment, the composition concentrate comprises the mixture of two different polyethylene glycol (PEG) polymers as defined herein in at total amount of 84-90% (v/v), and 10-16% (v/v) water. In one embodiment, the composition concentrate comprises the mixture of two different polyethylene glycol (PEG) polymers as defined herein in at total amount of 85-90% (v/v), and 10-15% (v/v) water. In one embodiment, the composition concentrate comprises the mixture of two different polyethylene glycol (PEG) polymers as defined herein in at total amount of 86-90% (v/v), and 10-14% (v/v) water. In one embodiment, the composition concentrate comprises the mixture of two different polyethylene glycol (PEG) polymers as defined herein in at total amount of 87-90% (v/v), and 10-13% (v/v) water. In one embodiment, the composition concentrate comprises the mixture of two different polyethylene glycol (PEG) polymers as defined herein in at total amount of 88-90% (v/v), and 10-12% (v/v) water. In one embodiment, the composition concentrate comprises the mixture of two different polyethylene glycol (PEG) polymers as defined herein in at total amount of 89-90% (v/v), and 10-11% (v/v) water. In one embodiment, the composition concentrate comprises the mixture of two different polyethylene glycol (PEG) polymers as defined herein in at total amount of 90% (v/v), and 10% (v/v) water.

In certain embodiments, the addition of a salt has a further stabilizing effect on the composition concentrates described herein, as shown in Example 7. Thus, in one embodiment, the composition concentrate further comprises a salt such as an inorganic salt containing a metal cation, especially a Group 1 or 2 metal cation. When included, the salt is typically present in the composition concentrate at a concentration of 1-50 mM. In one embodiment, the salt is sodium chloride, and is suitably present at a concentration of 20-40 mM, such as about 25 mM. In another embodiment, the salt is calcium chloride, and is suitably present at a concentration of 5-10 mM, such as about 5 mM.

In certain embodiments, the addition of a sugar has a further stabilizing effect on the composition concentrates described herein, as shown in Example 8. Thus, in one embodiment, the composition concentrate further comprises a sugar, which is suitably selected from the group consisting of glucose, sucrose, trehalose, raffinose, stachyose and verbascose, and is suitably sucrose. When included, the sugar is typically present in the composition concentrate at a concentration of 1-50 mM, e.g. 20-40 mM, such as about 25 mM.

In one embodiment, the composition concentrate further comprises an antioxidant, which is suitably selected from the group consisting of monothioglycerol, butylated hydroxyanisole, glutathione (reduced), and methionine, and is suitably monothioglycerol. When included, the antioxidant is typically present in the composition concentrate at a concentration of 1-10 mg/mL, such as 1-5 mg/mL e.g. 5 mg/mL. Monothioglycerol is also known as 1-thioglycerol, α-monothioglycerol, α-thioglycerol and 3-mercapto-1,2-propanediol.

In one embodiment the composition concentrate is substantially free of, or free of monothioglycerol.

The effect of pH on the stability of bendamustine composition concentrates is shown in Example 3, where a range of pH values between 3 and 9.25 were tested, with the optimal pH being observed to be between 3.0 and 4.0. Thus, in one embodiment, the pH of the composition concentrate of the invention is in the range 2.5 to 4.0, such as in the range 2.5 to 3.5, or is about 3.0. In embodiments where the composition concentrate does not comprise added buffer, the pH of the solution is maintained by the buffering capacity of bendamustine itself. If required, pH can be adjusted using hydrochloric acid or other suitable acid or sodium hydroxide or other suitable base. It should be noted that all references herein to “pH” refer to the pH of a composition concentrate evaluated at 21° C. All references to “pKₐ” refer to the pKₐ of an ionisable group evaluated at 25° C. (see CRC Handbook of Chemistry and Physics, 79^{th} Edition, 1998, D. R. Lide).

The composition concentrate may comprise one or more buffers, in particular buffers with suitable buffering capacity in the pH range 2.5 to 4.0, such as pH range 2.5 to 3.5. In one embodiment, the composition concentration comprises a buffer or buffers selected from the group consisting of citrate, formate, glycine, alanine, aspartate, malate, glyoxylate, gluconate, lactate, glycolate, tartrate and succinate. The skilled person will understand that buffers are present in solution at the target pH in an equilibrium between a protonated and deprotonated form. Hence the reference to “citrate” or “lactate” etc. as buffer will be understood to mean a mixture of that ion and the corresponding acid as buffer in a ratio according to the target pH. In this context, “target pH” means the pH at which the composition concentrate is intended to be buffered. At a pH below the pKa of the ionisable group the protonated form predominates. At a pH above the pKa of the ionisable group the deprotonated form predominates. A buffer is most effective at controlling (i.e. buffering) pH at pH values within 2 pH units or more suitably within around 1 pH unit of the pKa of the or an ionisable group of the buffer. The pKa of the ionisable carboxylic acid group of lactic acid is around 3.6. Consequently, lactate is a particularly suitable buffer in composition concentrates of the invention at a pH in the range between 2.5 and 4. When buffers are included in a composition concentrate of the invention, the buffer acid (e.g. citric acid, lactic acid etc.) may be added and the pH raised to the desired pH by addition of a base such as sodium hydroxide.

When included, the concentration of the buffer in the composition concentrate is typically in the range 1-50 mM such as 1-20 mM such as 2-15 mM such as 5-10 mM.

In one embodiment when the buffer is lactate the concentration of buffer in the composition concentrate may for example be 1-50 mM such 25-50 mM e.g. 40-50 mM.

In one embodiment, the composition concentrate is substantially free of, or free of organic small molecule solvents of molecular weight 50-120 Da. It should be noted that in the context of the present invention, monothioglycerol is used as an antioxidant and is not considered to be an organic small molecule solvent at the concentration used as an antioxidant.

In one embodiment, the composition concentrate is substantially free of, or free of propylene glycol.

In one embodiment, the composition concentrate is substantially free of, or free of N,N-dimethylacetamide, dimethyl sulfoxide, 1-methyl-2-pyrrolidinone, ethanol, isopropyl alcohol and benzyl alcohol. In one embodiment, the composition concentrate is substantially free of, or free of dimethyl sulfoxide (DMSO).

Preferably the composition concentrate is substantially free of, or free of ethanol. The advantages of avoiding ethanol are evident e.g. the presence of ethanol can create a fire risk in manufacture and is unacceptable for religious reasons in many cultures.

In one embodiment, the composition concentrate is substantially free of, or free of cyclodextrin. The term “cyclodextrin” is intended to cover all types of cyclodextrin.

“Substantially free of” as used herein means that the composition concentrate contains <0.1 mg/mL of the stated component if the component is a solid at 21° C., or <0.1% (v/v) if the component is a liquid at 21° C.

The composition concentrate may comprise a non-ionic surfactant, which is suitably selected from the group consisting of an alkyl glycoside, a polysorbate, an alkyl ether of polyethylene glycol, a block copolymer of polyethylene glycol and polypropylene glycol, and an alkylphenyl ether of polyethylene glycol.

When the non-ionic surfactant is an alkyl glycoside, it is suitably selected from the group consisting of dodecyl maltoside, dodecyl glucoside, octyl glucoside, octyl maltoside, decyl glucoside, decyl maltoside, decyl glucopyranoside, tridecyl glucoside, tridecyl maltoside, tetradecyl glucoside, tetradecyl maltoside, hexadecyl glucoside, hexadecyl maltoside, sucrose monooctanoate, sucrose monodecanoate, sucrose monododecanoate, sucrose monotridecanoate, sucrose monotetradecanoate and sucrose monohexadecanoate. In one embodiment, the non-ionic surfactant is dodecyl maltoside or decyl glucopyranoside, and in particular is dodecyl maltoside.

When the non the non-ionic surfactant is a polysorbate, it is suitably selected from the group consisting of polysorbate 20, polysorbate 40, polysorbate 60 or polysorbate 80. In one embodiment, the non-ionic surfactant is polysorbate 20 or polysorbate 80. Polysorbates are known under a range of brand names including in particular Tween “XX”, and also Alkest TW “XX”, where “XX” is 20, 40, 60 or 80.

When the non-ionic surfactant is an alkyl ether of polyethylene glycol, it is suitably selected from the group consisting of polyethylene glycol (2) hexadecyl ether (Brij 52), polyethylene glycol (2) oleyl ether (Brij 93), polyethylene glycol (2) dodecyl ether (Brij L4), polyethylene glycol (4) lauryl ether (Brij 30), polyethylene glycol (10) lauryl ether (Brij 35), polyethylene glycol (20) hexadecyl ether (Brij 58) and polyethylene glycol (10) stearyl ether (Brij 78).

When the non-ionic surfactant is a block copolymer of polyethylene glycol and polypropylene glycol, it is suitably selected from the group consisting of poloxamer 188, poloxamer 407, poloxamer 171 or poloxamer 185. Poloxamers are also known under brand names Pluronics or Koliphors. For example, poloxamer 188 is marketed as Pluronic F-68.

When the non-ionic surfactant is an alkylphenyl ether of polyethylene glycol it is suitably 4-(1,1,3,3-tetramethylbutyl)phenyl-polyethylene glycol, also known under a brand name Triton X-100.

When included, the concentration of the non-ionic surfactant in the composition concentrate is typically in the range 1-5000 μg/mL, 1-1000 μg/mL, such as 5-500 μg/mL, 10-400 μg/mL, 20-400 μg/mL, 50-400 μg/mL, 10-300 μg/mL, 20-300 μg/mL, 50-300 μg/mL, 10-200 μg/mL, 20-200 μg/mL, 50-200 μg/mL, 10-100 μg/mL, 20-100 μg/mL, 50-100 μg/mL or around 50 μg/mL.

The composition concentrates of the invention may additionally comprise a preservative such as a phenolic or a benzylic preservative. The preservative is suitably selected from the group consisting of phenol, m-cresol, chlorocresol, benzyl alcohol, propyl paraben and methyl paraben, in particular phenol, m-cresol and benzyl alcohol, and mixtures thereof. When included, the concentration of preservative is typically 10-100 mM, for example 20-80 mM, such as 25-50 mM. The optimal concentration of the preservative in the composition is selected to ensure the composition passes the Pharmacopoeia Antimicrobial Effectiveness Test (USP <51>, Vol. 32).

In one embodiment, there is provided a storage stable liquid composition concentrate comprising bendamustine or a pharmaceutically acceptable salt thereof and a liquid solvent in which said bendamustine or a pharmaceutically acceptable salt thereof is dissolved;
wherein said composition consists of or consists essentially of:
(i) bendamustine or a pharmaceutically acceptable salt thereof (e.g. at a concentration of 45-75 mM, such as about 61 mM);
(ii) a mixture of two different polyethylene glycol (PEG) polymers selected from the group consisting of PEG200, PEG300, PEG400 and PEG600;
(iii) 5-20% (v/v) of water;
(iv) optionally an inorganic salt containing a metal cation, especially a Group 1 or 2 metal cation (e.g. sodium chloride or calcium chloride, in particular sodium chloride) e.g. at a concentration of 1-50 mM;
(v) optionally a sugar (e.g. glucose, sucrose, trehalose, raffinose, stachyose or verbascose, in particular sucrose) e.g. at a concentration of 1-50 mM; (vi) optionally an antioxidant (e.g. monothioglycerol, butylated hydroxyanisole, glutathione (reduced) or methionine, in particular monothioglycerol) e.g. at a concentration of 1-10 mg/mL;
(vii) optionally a non-ionic surfactant;
(viii) optionally a preservative; and
(ix) optionally a buffer;
wherein the pH of the composition concentrate is in the range 2.5-4.0 (e.g. in the range 2.5-3.5). In one embodiment, the liquid composition concentrate is provided in a vial (e.g. a glass vial).

In one embodiment, there is provided a storage stable liquid composition concentrate comprising bendamustine or a pharmaceutically acceptable salt thereof and a liquid solvent in which said bendamustine or a pharmaceutically acceptable salt thereof is dissolved;
wherein said composition consists of or consists essentially of:
(i) bendamustine or a pharmaceutically acceptable salt thereof (e.g. at a concentration of 45-75 mM, such as about 61 mM);
(ii) a mixture of two different polyethylene glycol (PEG) polymers selected from the group consisting of PEG200, PEG300, PEG400 and PEG600;
(iii) 5-20% (v/v) of water;
(iv) optionally an inorganic salt containing a metal cation, especially a Group 1 or 2 metal cation (e.g. sodium chloride or calcium chloride, in particular sodium chloride) e.g. at a concentration of 1-50 mM;
(v) optionally a sugar (e.g. glucose, sucrose, trehalose, raffinose, stachyose or verbascose, in particular sucrose) e.g. at a concentration of 1-50 mM; (vi) optionally an antioxidant (e.g. monothioglycerol, butylated hydroxyanisole, glutathione (reduced) or methionine, in particular monothioglycerol) e.g. at a concentration of 1-10 mg/mL;
(vii) optionally a non-ionic surfactant;
(viii) optionally a preservative; and
(ix) optionally a buffer;
wherein the pH of the composition concentrate is in the range 2.5-4.0 (e.g. in the range 2.5-3.5); and
wherein in the mixture of two different polyethylene glycol (PEG) polymers, the polyethylene glycol polymer with lower average molecular weight is present in an equal or a greater proportion (especially a greater proportion) than the other polyethylene glycol polymer (% v/v). In one embodiment, the liquid composition concentrate is provided in a vial (e.g. a glass vial).

In one embodiment, there is provided a storage stable liquid composition concentrate comprising bendamustine or a pharmaceutically acceptable salt thereof and a liquid solvent in which said bendamustine or a pharmaceutically acceptable salt thereof is dissolved;
wherein said composition consists of or consists essentially of:
(i) bendamustine or a pharmaceutically acceptable salt thereof (e.g. at a concentration of 45-75 mM, such as about 61 mM);
(ii) a mixture of two different polyethylene glycol (PEG) polymers selected from the group consisting of PEG200, PEG300, PEG400 and PEG600; wherein said mixture is present in a total concentration of 80-95% (v/v);
(iii) 5-20% (v/v) of water;
(iv) optionally an inorganic salt containing a metal cation, especially a Group 1 or 2 metal cation (e.g. sodium chloride or calcium chloride, in particular sodium chloride) e.g. at a concentration of 1-50 mM;
(v) optionally a sugar (e.g. glucose, sucrose, trehalose, raffinose, stachyose or verbascose, in particular sucrose) e.g. at a concentration of 1-50 mM;
(vi) optionally an antioxidant (e.g. monothioglycerol, butylated hydroxyanisole, glutathione (reduced) or methionine, in particular monothioglycerol) e.g. at a concentration of 1-10 mg/mL;
(vii) optionally a non-ionic surfactant;
(viii) optionally a preservative; and
(ix) optionally a buffer;
wherein the pH of the composition concentrate is in the range 2.5-4.0 (e.g. in the range 2.5-3.5). In one embodiment, the liquid composition concentrate is provided in a vial (e.g. a glass vial).

In one embodiment, there is provided a storage stable liquid composition concentrate comprising bendamustine or a pharmaceutically acceptable salt thereof and a liquid solvent in which said bendamustine or a pharmaceutically acceptable salt thereof is dissolved;
wherein said composition consists of or consists essentially of:
(i) bendamustine or a pharmaceutically acceptable salt thereof (e.g. at a concentration of 45-75 mM, such as about 61 mM);
(ii) a mixture of two different polyethylene glycol (PEG) polymers selected from the group consisting of PEG200, PEG300, PEG400 and PEG600; wherein said mixture is present in a total concentration of 80-95% (v/v) and
(iii) 5-20% (v/v) of water;
(iv) optionally an inorganic salt containing a metal cation, especially a Group 1 or 2 metal cation (e.g. sodium chloride or calcium chloride, in particular sodium chloride) e.g. at a concentration of 1-50 mM;
(v) optionally a sugar (e.g. glucose, sucrose, trehalose, raffinose, stachyose or verbascose, in particular sucrose) e.g. at a concentration of 1-50 mM;
(vi) optionally an antioxidant (e.g. monothioglycerol, butylated hydroxyanisole, glutathione (reduced) or methionine, in particular monothioglycerol) e.g. at a concentration of 1-10 mg/mL;
(vii) optionally a non-ionic surfactant;
(viii) optionally a preservative; and
(ix) optionally a buffer;
wherein the pH of the composition concentrate is in the range 2.5-4.0 (e.g. in the range 2.5-3.5); and
wherein in the mixture of two different polyethylene glycol (PEG) polymers, the polyethylene glycol polymer with lower average molecular weight is present in an equal or a greater proportion (especially a greater proportion) than the other polyethylene glycol polymer (% v/v). In one embodiment, the liquid composition concentrate is provided in a vial (e.g. a glass vial).

In one embodiment, there is provided a storage stable liquid composition concentrate comprising bendamustine or a pharmaceutically acceptable salt thereof and a liquid solvent in which said bendamustine or a pharmaceutically acceptable salt thereof is dissolved;
wherein said composition consists of or consists essentially of:
(i) bendamustine or a pharmaceutically acceptable salt thereof (e.g. at a concentration of 45-75 mM, such as about 61 mM);
(ii) a mixture of two different polyethylene glycol (PEG) polymers selected from the group consisting of PEG200, PEG300, PEG400 and PEG600;
(iii) 5-20% (v/v) of water;
(iv) optionally an inorganic salt containing a metal cation (e.g. sodium chloride or calcium chloride, in particular sodium chloride) e.g. at a concentration of 1-50 mM;
(v) optionally a sugar (e.g. glucose, sucrose, trehalose, raffinose, stachyose or verbascose, in particular sucrose) e.g. at a concentration of 1-50 mM; and
(vi) optionally an antioxidant (e.g. monothioglycerol, butylated hydroxyanisole, glutathione (reduced) or methionine, in particular monothioglycerol) e.g. at a concentration of 1-10 mg/mL;
wherein the pH of the composition concentrate is in the range 2.5-4.0 (e.g. in the range 2.5-3.5). In one embodiment, the liquid composition concentrate is provided in a vial (e.g. a glass vial).

In one embodiment, there is provided a storage stable liquid composition concentrate comprising bendamustine or a pharmaceutically acceptable salt thereof and a liquid solvent in which said bendamustine or a pharmaceutically acceptable salt thereof is dissolved;
wherein said composition consists of or consists essentially of:
(i) bendamustine or a pharmaceutically acceptable salt thereof (e.g. at a concentration of 45-75 mM, such as about 61 mM);
(ii) a mixture of two different polyethylene glycol (PEG) polymers selected from the group consisting of PEG200, PEG300, PEG400 and PEG600;
(iii) 5-20% (v/v) of water;
(iv) optionally an inorganic salt containing a metal cation, especially a Group 1 or 2 metal cation (e.g. sodium chloride or calcium chloride, in particular sodium chloride) e.g. at a concentration of 1-50 mM;
(v) optionally a sugar (e.g. glucose, sucrose, trehalose, raffinose, stachyose or verbascose, in particular sucrose) e.g. at a concentration of 1-50 mM; and
(vi) optionally an antioxidant (e.g. monothioglycerol, butylated hydroxyanisole, glutathione (reduced) or methionine, in particular monothioglycerol) e.g. at a concentration of 1-10 mg/mL;
wherein the pH of the composition concentrate is in the range 2.5-4.0 (e.g. in the range 2.5-3.5); and
wherein in the mixture of two different polyethylene glycol (PEG) polymers, the polyethylene glycol polymer with lower average molecular weight is present in an equal or a greater proportion (especially a greater proportion) than the other polyethylene glycol polymer (% v/v). In one embodiment, the liquid composition concentrate is provided in a vial (e.g. a glass vial).

In one embodiment, there is provided a storage stable liquid composition concentrate comprising bendamustine or a pharmaceutically acceptable salt thereof and a liquid solvent in which said bendamustine or a pharmaceutically acceptable salt thereof is dissolved;
wherein said composition consists of or consists essentially of:
(i) bendamustine or a pharmaceutically acceptable salt thereof (e.g. at a concentration of 45-75 mM, such as about 61 mM);
(ii) a mixture of two different polyethylene glycol (PEG) polymers selected from the group consisting of PEG200, PEG300, PEG400 and PEG600; wherein said mixture is present in a total concentration of 80-95% (v/v) and
(iii) 5-20% (v/v) of water;
(iv) optionally an inorganic salt containing a metal cation, especially a Group 1 or 2 metal cation (e.g. sodium chloride or calcium chloride, in particular sodium chloride) e.g. at a concentration of 1-50 mM;
(v) optionally a sugar (e.g. glucose, sucrose, trehalose, raffinose, stachyose or verbascose, in particular sucrose) e.g. at a concentration of 1-50 mM;
(vi) optionally an antioxidant (e.g. monothioglycerol, butylated hydroxyanisole, glutathione (reduced) or methionine, in particular monothioglycerol) e.g. at a concentration of 1-10 mg/mL;
wherein the pH of the composition concentrate is in the range 2.5-4.0 (e.g. in the range 2.5-3.5). In one embodiment, the liquid composition concentrate is provided in a vial (e.g. a glass vial).

In one embodiment, there is provided a storage stable liquid composition concentrate comprising bendamustine or a pharmaceutically acceptable salt thereof and a liquid solvent in which said bendamustine or a pharmaceutically acceptable salt thereof is dissolved;
wherein said composition consists of or consists essentially of:
(i) bendamustine or a pharmaceutically acceptable salt thereof (e.g. at a concentration of 45-75 mM, such as about 61 mM);
(ii) a mixture of two different polyethylene glycol (PEG) polymers selected from the group consisting of PEG200, PEG300, PEG400 and PEG600; wherein said mixture is present in a total concentration of 80-95% (v/v) and
(iii) 5-20% (v/v) of water;
(iv) an antioxidant (e.g. monothioglycerol, butylated hydroxyanisole, glutathione (reduced) or methionine, in particular monothioglycerol) e.g. at a concentration of 1-10 mg/mL; and
(v) an inorganic salt containing a metal cation, especially a Group 1 or 2 metal cation (e.g. sodium chloride or calcium chloride, in particular sodium chloride) e.g. at a concentration of 1-50 mM;
wherein the pH of the composition concentrate is in the range 2.5-4.0 (e.g. in the range 2.5-3.5); and
wherein in the mixture of two different polyethylene glycol (PEG) polymers, the polyethylene glycol polymer with lower average molecular weight is suitably present in an equal or a greater proportion (especially a greater proportion) than the other polyethylene glycol polymer (% v/v). In one embodiment, the liquid composition concentrate is provided in a vial (e.g. a glass vial).

In one embodiment, there is provided a storage stable liquid composition concentrate comprising bendamustine or a pharmaceutically acceptable salt thereof and a liquid solvent in which said bendamustine or a pharmaceutically acceptable salt thereof is dissolved;
wherein said composition consists of or consists essentially of:
(i) bendamustine or a pharmaceutically acceptable salt thereof (e.g. at a concentration of 45-75 mM, such as about 61 mM);
(ii) a mixture of two different polyethylene glycol (PEG) polymers selected from the group consisting of PEG200, PEG300, PEG400 and PEG600; wherein said mixture is present in a total concentration of 80-95% (v/v) and
(iii) 5-20% (v/v) of water;
(iv) an antioxidant (e.g. monothioglycerol, butylated hydroxyanisole, glutathione (reduced) or methionine, in particular monothioglycerol) e.g. at a concentration of 1-10 mg/mL; and
(v) a sugar (e.g. glucose, sucrose, trehalose, raffinose, stachyose or verbascose, in particular sucrose) e.g. at a concentration of 1-50 mM;
wherein the pH of the composition concentrate is in the range 2.5-4.0 (e.g. in the range 2.5-3.5); and
wherein in the mixture of two different polyethylene glycol (PEG) polymers, the polyethylene glycol polymer with lower average molecular weight is suitably present in an equal or a greater proportion (especially a greater proportion) than the other polyethylene glycol polymer (% v/v). In one embodiment, the liquid composition concentrate is provided in a vial (e.g. a glass vial).

In one embodiment, there is provided a storage stable liquid composition concentrate comprising bendamustine or a pharmaceutically acceptable salt thereof and a liquid solvent in which said bendamustine or a pharmaceutically acceptable salt thereof is dissolved;
wherein said composition consists of or consists essentially of:
(i) bendamustine or a pharmaceutically acceptable salt thereof (e.g. at a concentration of 45-75 mM, such as about 61 mM);
(ii) a mixture of PEG300 and PEG400, wherein said mixture is present in a total concentration of 80-95% (e.g. 90%) (v/v);
(iii) 5-20% (e.g. 10%) (v/v) of water;
(iv) optionally an inorganic salt containing a metal cation, especially a Group 1 or 2 metal cation (e.g. sodium chloride or calcium chloride, in particular sodium chloride) e.g. at a concentration of 1-50 mM;
(v) optionally a sugar (e.g. glucose, sucrose, trehalose, raffinose, stachyose or verbascose, in particular sucrose) e.g. at a concentration of 1-50 mM;
(vi) optionally an antioxidant (e.g. monothioglycerol, butylated hydroxyanisole, glutathione (reduced) or methionine, in particular monothioglycerol) e.g. at a concentration of 1-10 mg/mL, such as 5 mg/mL;
(vii) optionally a non-ionic surfactant;
(viii) optionally a preservative; and
(ix) optionally a buffer;
wherein the pH of the composition concentrate is in the range 2.5-4.0 (e.g. in the range 2.5-3.5); and
wherein in the mixture of PEG300 and PEG400, the PEG300 is present in an equal or a greater proportion (% v/v) than the PEG400. In a further embodiment, the PEG300 is present in a greater proportion than the PEG400 (% v/v). In one embodiment, the liquid composition concentrate is provided in a vial (e.g. a glass vial).

In one embodiment, there is provided a storage stable liquid composition concentrate comprising bendamustine or a pharmaceutically acceptable salt thereof and a liquid solvent in which said bendamustine or a pharmaceutically acceptable salt thereof is dissolved;
wherein said composition consists of or consists essentially of:
(i) bendamustine or a pharmaceutically acceptable salt thereof (e.g. at a concentration of 45-75 mM, such as about 61 mM);
(ii) a mixture of PEG300 and PEG400, wherein said mixture is present in a total concentration of 80-95% (e.g. 90%) (v/v);
(iii) 5-20% (e.g. 10%) (v/v) of water;
(iv) sodium chloride at a concentration of 10-50 mM (e.g. 20-40 mM or 25 mM);
(v) optionally a sugar (e.g. glucose, sucrose, trehalose, raffinose, stachyose or verbascose, in particular sucrose) e.g. at a concentration of 1-50 mM;
(vi) optionally an antioxidant (e.g. monothioglycerol, butylated hydroxyanisole, glutathione (reduced) or methionine, in particular monothioglycerol) e.g. at a concentration of 1-10 mg/mL, such as 5 mg/mL;
(vii) optionally a non-ionic surfactant;
(viii) optionally a preservative; and
(ix) optionally a buffer;
wherein the pH of the composition concentrate is in the range 2.5-4.0 (e.g. in the range 2.5-3.5); and
wherein in the mixture of PEG300 and PEG400, the PEG300 is present in an equal or a greater proportion (% v/v) than the PEG400. In a further embodiment, the PEG300 is present in a greater proportion than the PEG400 (% v/v). In one embodiment, the liquid composition concentrate is provided in a vial (e.g. a glass vial).

In one embodiment, there is provided a storage stable liquid composition concentrate comprising bendamustine or a pharmaceutically acceptable salt thereof and a liquid solvent in which said bendamustine or a pharmaceutically acceptable salt thereof is dissolved;
wherein said composition consists of or consists essentially of:
(i) bendamustine or a pharmaceutically acceptable salt thereof (e.g. at a concentration of 45-75 mM, such as about 61 mM);
(ii) a mixture of PEG300 and PEG400, wherein said mixture is present in a total concentration of 80-95% (e.g. 90%) (v/v);
(iii) 5-20% (e.g. 10%) (v/v) of water;
(iv) sodium chloride at a concentration of 10-50 mM (e.g. 20-40 mM or 25 mM); and
(v) an antioxidant (e.g. monothioglycerol, butylated hydroxyanisole, glutathione (reduced) or methionine, in particular monothioglycerol) e.g. at a concentration of 1-10 mg/mL, such as 5 mg/mL;
(vi) optionally a sugar (e.g. glucose, sucrose, trehalose, raffinose, stachyose or verbascose, in particular sucrose) e.g. at a concentration of 1-50 mM;
(vii) optionally a non-ionic surfactant;
(viii) optionally a preservative; and
(ix) optionally a buffer;
wherein the pH of the composition concentrate is in the range 2.5-4.0 (e.g. in the range 2.5-3.5); and
wherein in the mixture of PEG300 and PEG400, the PEG300 is present in an equal or a greater proportion (% v/v) than the PEG400. In a further embodiment, the PEG300 is present in a greater proportion than the PEG400 (% v/v). In one embodiment, the antioxidant is monothioglycerol. In a further embodiment the monothioglycerol is at a concentration of 1-10 mg/ml (e.g. 5 mg/ml). In one embodiment, the liquid composition concentrate is provided in a vial (e.g. a glass vial).

In one embodiment, there is provided a storage stable liquid composition concentrate comprising bendamustine or a pharmaceutically acceptable salt thereof and a liquid solvent in which said bendamustine or a pharmaceutically acceptable salt thereof is dissolved;
wherein said composition consists of or consists essentially of:
(i) bendamustine or a pharmaceutically acceptable salt thereof (e.g. at a concentration of 45-75 mM, such as about 61 mM);
(ii) a mixture of PEG300 and PEG400, wherein said mixture is present in a total concentration of 80-95% (e.g. 90%) (v/v);
(iii) 5-20% (e.g. 10%) (v/v) of water;
(iv) a sugar (e.g. glucose, sucrose, trehalose, raffinose, stachyose or verbascose, in particular sucrose) e.g. at a concentration of 1-50 mM;
(v) optionally an inorganic salt containing a metal cation, especially a Group 1 or 2 metal cation (e.g. sodium chloride or calcium chloride, in particular sodium chloride) e.g. at a concentration of 1-50 mM;
(vi) optionally an antioxidant (e.g. monothioglycerol, butylated hydroxyanisole, glutathione (reduced) or methionine, in particular monothioglycerol) e.g. at a concentration of 1-10 mg/mL, such as 5 mg/mL;
(vii) optionally a non-ionic surfactant;
(viii) optionally a preservative; and
(ix) optionally a buffer;
wherein the pH of the composition concentrate is in the range 2.5-4.0 (e.g. in the range 2.5-3.5); and
wherein in the mixture of PEG300 and PEG400, the PEG300 is present in an equal or a greater proportion (% v/v) than the PEG400. In a further embodiment, the PEG300 is present in a greater proportion than the PEG400 (% v/v). In one embodiment, the liquid composition concentrate is provided in a vial (e.g. a glass vial).

In one embodiment, there is provided a storage stable liquid composition concentrate comprising bendamustine or a pharmaceutically acceptable salt thereof and a liquid solvent in which said bendamustine or a pharmaceutically acceptable salt thereof is dissolved;
wherein said composition consists of or consists essentially of:
(i) bendamustine or a pharmaceutically acceptable salt thereof (e.g. at a concentration of 45-75 mM, such as about 61 mM);
(ii) a mixture of PEG300 and PEG400, wherein said mixture is present in a total concentration of 80-95% (e.g. 90%) (v/v);
(iii) 5-20% (e.g. 10%) (v/v) of water;
(iv) a sugar (e.g. glucose, sucrose, trehalose, raffinose, stachyose or verbascose, in particular sucrose) e.g. at a concentration of 1-50 mM;
(v) an antioxidant (e.g. monothioglycerol, butylated hydroxyanisole, glutathione (reduced) or methionine, in particular monothioglycerol) e.g. at a concentration of 1-10 mg/mL, such as 5 mg/mL;
(vi) optionally an inorganic salt containing a metal cation, especially a Group 1 or 2 metal cation (e.g. sodium chloride or calcium chloride, in particular sodium chloride) e.g. at a concentration of 1-50 mM;
(vii) optionally a non-ionic surfactant;
(viii) optionally a preservative; and
(ix) optionally a buffer;
wherein the pH of the composition concentrate is in the range 2.5-4.0 (e.g. in the range 2.5-3.5); and
wherein in the mixture of PEG300 and PEG400, the PEG300 is present in an equal or a greater proportion (% v/v) than the PEG400. In a further embodiment, the PEG300 is present in a greater proportion than the PEG400 (% v/v). In one embodiment, the antioxidant is monothioglycerol. In a further embodiment the monothioglycerol is at a concentration of 1-10 mg/ml (e.g. 5 mg/ml). In one embodiment, the liquid composition concentrate is provided in a vial (e.g. a glass vial).

In one embodiment, there is provided a storage stable liquid composition concentrate comprising bendamustine or a pharmaceutically acceptable salt thereof and a liquid solvent in which said bendamustine or a pharmaceutically acceptable salt thereof is dissolved;
wherein said composition consists of or consists essentially of:
(i) bendamustine or a pharmaceutically acceptable salt thereof (e.g. at a concentration of 45-75 mM, such as about 61 mM);
(ii) a mixture of two or more different polyethylene glycol (PEG) polymers, wherein each polyethylene glycol has an average molecular weight in the range 150-650 Da for example a mixture of PEG300 and PEG400, wherein said mixture is present in a total concentration of 80-95% (e.g. 90%) (v/v); (iii) 5-20% (e.g. 10%) (v/v) of water;
(iv) optionally a sugar (e.g. glucose, sucrose, trehalose, raffinose, stachyose or verbascose, in particular sucrose) e.g. at a concentration of 1-50 mM; (v) a buffer, especially lactate;
(vi) optionally an inorganic salt containing a metal cation, especially a Group 1 or 2 metal cation (e.g. sodium chloride or calcium chloride, in particular sodium chloride) e.g. at a concentration of 1-50 mM;
(vii) optionally a non-ionic surfactant; and
(viii) optionally a preservative; and
wherein the pH of the composition concentrate is in the range 2.5-4.0 (e.g. in the range 2.5-3.5). In one embodiment the mixture of two or more different polyethylene glycol (PEG) polymers is a mixture of PEG300 and PEG400 and the PEG300 is present in an equal or a greater proportion than the PEG400 (% v/v). In a further embodiment, the PEG300 is present in a greater proportion than the PEG400 (% v/v). In one embodiment the buffer is lactate present at a concentration of 1-50 mM e.g. 25-50 mM e.g. 40-50 mM. In one embodiment, the liquid composition concentrate is provided in a vial (e.g. a glass vial).

As used herein when referring to the composition of the invention, the term “consists of” means that no further components are included in the composition other than those listed. As used herein when referring to the composition of the invention, the term “consists essentially of” means that specific further components can be present, but said components do not materially affect the essential characteristics of the composition, and are typically present in a de minimis amount.

Suitably the composition concentrate of the invention remains as a clear solution (e.g. as measured according to the Visual Assessment Method in the General Methods) following storage at 2-8° C. for an extended period of time, such as at least 26 weeks, at least 12 months, or at least 18 months.

Suitably the composition concentrate of the invention remains as a clear solution (e.g. as measured according to the Visual Assessment Method in the General Methods) following storage at 30° C. for at least 1 day, such as at least 3 days, at least 1 week or at least 4 weeks.

Suitably the composition concentrate of the invention comprises no more than 5% total impurities, such as no more than 4%, such as no more than 3%, such as no more than 2% total impurities (by total weight of bendamustine in the composition (e.g. as measured by using the RP-HPLC method described in General Methods)), following storage at 2-8° C. for an extended period of time, such as at least 26 weeks, at least 12 months, or at least 18 months.

Suitably the composition concentrate of the invention comprises no more than 5% total impurities, such as no more than 4%, such as no more than 3%, such as no more than 2% total impurities (by total weight of bendamustine in the composition (e.g. as measured by using the RP-HPLC method described in General Methods)), following storage at 30° C. for at least 1 day, such as at least 3 days, at least 1 week or at least 4 weeks.

Suitably the composition concentrate of the invention has comparable or improved storage stability in particular physical stability (e.g. as measured according to the Visual Assessment Method in the General Methods) and has lower viscosity (e.g. as measured according to Viscosity Assessment Method in the General Methods) than the commercially available formulation of Bendeka® (Formulation 7 of Example 4), following storage at 2-8° C. for an extended period of time, such as at least 26 weeks, at least 12 months, or at least 18 months.

Suitably the composition concentrate of the invention has comparable or improved storage stability in particular chemical stability (e.g. using the RP-HPLC method described General Methods)) and has lower viscosity (e.g. as measured according to Viscosity Assessment Method in the General Methods) than the commercially available formulation of Bendeka® (Formulation 7 of Example 4), following storage at 30° C. for at least 1 day, such as at least 3 days, at least 1 week or at least 4 weeks.

Suitably the composition concentrate of the invention comprises a comparable or lower level of impurities (by total weight of bendamustine in the composition (e.g. as measured by using the RP-HPLC method described in General Methods)) and has lower viscosity (e.g. as measured according to Viscosity Assessment Method in the General Methods) than the commercially available formulation of Bendeka® (Formulation 7 of Example 4) following storage at 2-8° C. for an extended period of time, such as at least 26 weeks, at least 12 months, or at least 18 months.

Suitably the composition concentrate of the invention comprises a comparable or lower level of impurities (by total weight of bendamustine in the composition (e.g. as measured by using the RP-HPLC method described in General Methods)) and has lower viscosity (e.g. as measured according to Viscosity Assessment Method in the General Methods) than the commercially available formulation of Bendeka® (Formulation 7 of Example 4) following storage at 30° C. for at least 1 day, such as at least 3 days, at least 1 week or at least 4 weeks.

In one embodiment, the composition concentrate of the invention is a composition for use in therapy. In one embodiment, the composition concentrate of the invention is a pharmaceutical composition.

Bendamustine is indicated inter alia for the treatment of chronic lymphocytic leukaemia (CLL), multiple myeloma and non-Hodgkin lymphoma (NHL) (in particular indolent B-cell non-Hodgkin lymphoma).

Accordingly, in one embodiment is provided a composition concentrate as described hereinabove in a diluted form for use in treating of chronic lymphocytic leukaemia (CLL), multiple myeloma or non-Hodgkin lymphoma (NHL). In one embodiment is provided a composition concentrate as described hereinabove in a diluted form for the manufacture of a medicament for treating chronic lymphocytic leukaemia (CLL), multiple myeloma or non-Hodgkin lymphoma (NHL). In one embodiment is provided a method of treating chronic lymphocytic leukaemia (CLL), multiple myeloma or non-Hodgkin lymphoma (NHL) which comprises administering to a patient, particularly a human patient, in need thereof a therapeutically effective amount of a diluted form of a composition concentrate as described hereinabove.

The bendamustine composition concentrates of the present invention can be used as a sole therapy, or in combination with other therapeutic agents. In one embodiment, the further therapeutic agent is selected from the group consisting of etoposide, rituximab, fludarabine, mitoxantrone, methotrexate, prednisone, vincristine and ⁹⁰Y-ibritumomab tiuxetan.

All embodiments described above with respect to the aqueous liquid composition concentrate apply equally to methods and uses of the invention.

There is also provided a container, in particular a vial which is suitably made of plastics or glass, containing one dose or a plurality of doses of the composition concentrate as described hereinabove. Suitably the container is a glass vial, in particular a type 1 glass vial e.g. containing 80% silica and 10% boric oxide, with a small amount of sodium oxide and aluminium oxide. In one embodiment is provided a multiple-dose vial, in particular a glass vial, containing the composition concentrate as described hereinabove.

In one embodiment, the fill volume of the vial is a 0.5 mL. In another embodiment, the fill volume of the vial is a 2 mL. In another embodiment, the fill volume of the vial is a 4 mL. In another embodiment, the fill volume of the vial is a 8 mL. In another embodiment, the fill volume of the vial is a 10 mL. In another embodiment, the fill volume of the vial is a 14 mL. Suitably, the fill volume of the vial is 4 mL. Typically, a vial with a larger fill volume is preferred to enable use of fewest number of vials for preparation of a dose.

The composition concentrate of the present invention is a ready-to-dilute liquid solution, meaning that it must be diluted prior to administration. Once diluted, the resulting solution (ready-to-administer solution) is usually administered by intravenous infusion. A typical method for administration of the composition concentrate of the invention involves transferring a volume of ready-to-dilute liquid solution to an infusion bag (sometimes known as an IV bag), where the volume of ready-to-dilute liquid solution is calculated according to the required dose and the volume of the IV bag. Following thorough mixing of the diluted solution, the contents of the bag can be administered to the patient.

The composition concentrate of the invention can be diluted in a pharmaceutically acceptable diluent such as isotonic saline (0.9% w/v), isotonic dextrose (5% w/v), isotonic mixtures of saline and dextrose (e.g. saline (0.45% w/v) and dextrose (2.5% w/v)), sterile water for injection or bacteriostatic water for injection.

Suitably, the composition concentrate of the invention (ready-to-dilute solution) is diluted in a suitable diluent by mixing 1 volume part of the composition concentrate with 3-13 volume parts of the suitable diluent. The concentration of bendamustine or pharmaceutically acceptable salt thereof in the resulting ready-to-administer solution is thus 4-14 times lower than that in the original ready-to-use solution. In one embodiment, the concentration of bendamustine or pharmaceutically acceptable salt thereof in the diluted composition (ready-to-administer) is 7-25% of the concentration in the composition concentrate. In one embodiment, the diluted composition (ready-to-administer) contains 1-60 mM, such as 1-50 mM, 1-25 mM, 2-25 mM, 3-20 mM, 4-15 mM or 4.5-13.6 mM bendamustine (optionally in the form of a pharmaceutically acceptable salt e.g. the hydrochloride salt).

Suitably the diluted form of the composition is free of visible particles and a colourless to slightly yellow solution. In one embodiment, the diluted form of the composition is isotonic. In another embodiment, the diluted form of the composition is slightly hypertonic. Typically, the diluted form is slightly hypertonic. Suitably the diluted form of the composition is stable for at least 24 hours at 2-8° C. and at least 3 hours at room temperature and room light.

Thus, in one embodiment is provided a method of preparing a solution for intravenous administration (ready-to-administer solution) by diluting a composition concentrate as described hereinabove in a diluent selected from isotonic saline (0.9% w/v), isotonic dextrose (5% w/v), isotonic mixtures of saline and dextrose (e.g. saline (0.45% w/v) and dextrose (2.5% w/v)), sterile water for injection or bacteriostatic water for injection In one embodiment, the method provides a diluted composition comprising 4.5-13.6 mM bendamustine (e.g. 1.85-5.6 mg/mL bendamustine hydrochloride).

### Additional Clauses of the Invention

1. A storage stable liquid composition concentrate comprising bendamustine or a pharmaceutically acceptable salt thereof and a liquid solvent in which said bendamustine or a pharmaceutically acceptable salt thereof is dissolved;
   wherein said composition comprises:
   (i) a mixture of two or more different polyethylene glycol (PEG) polymers, wherein each polyethylene glycol has an average molecular weight in the range 150-650 Da; and
   (ii) 5-20% (v/v) of water.
2. The storage stable liquid composition concentrate according to clause 1, wherein the bendamustine form is bendamustine hydrochloride.
3. The storage stable liquid composition concentrate according to clause 1 or clause 2, wherein the concentration of bendamustine or pharmaceutically acceptable salt thereof is 25-250 mM, e.g. 25-225 mM, 25-200 mM, 25-175 mM, 25-150 mM, 25-125 mM, 25-100 mM, 25-75 mM, 35-250 mM, 35-225 mM, 35-200 mM, 35-175 mM, 35-150 mM, 35-125 mM, 35-100 mM, 35-75 mM, 45-250 mM, 45-225 mM, 45-200 mM, 45-175 mM, 45-150 mM, 45-125 mM, 45-100 mM, or 45-75 mM, such as about 61 mM.
4. The storage stable liquid composition concentrate according to any one of clauses 1 to 3, wherein the composition concentrate comprises two different polyethylene glycol (PEG) polymers, wherein each polyethylene glycol has an average molecular weight in the range 150-650 Da.
5. The storage stable liquid composition concentrate according to clause 4, wherein the two different polyethylene glycol (PEG) polymers are selected from the group consisting of PEG200, PEG300, PEG400 and PEG600; and are suitably selected from the group consisting of PEG200, PEG300 and PEG400.
6. The storage stable liquid composition concentrate according to clause 5, wherein the two different polyethylene glycol polymers are PEG300 and PEG400.
7. The storage stable liquid composition concentrate according to any one of clauses 4 to 6, wherein in the mixture of two different polyethylene glycol (PEG) polymers, the polyethylene glycol polymer with lower average molecular weight is present in an equal or a greater proportion (especially a greater proportion) than the other polyethylene glycol polymer (% v/v).
8. The storage stable liquid composition concentrate according to clause 7, wherein the ratio of lower average molecular weight PEG to higher average molecular weight PEG is between 1.5:1 and 10:1 (% v/v), e.g. between 3:1 and 8:1, such as about 7:1.
9. The storage stable liquid composition concentrate according to any one of clauses 1 to 8, wherein the mixture of two or more different polyethylene glycol (PEG) polymers is present in a total amount of 80-95% (v/v).
10. The storage stable liquid composition concentrate according to any one of clauses 1 to 9, wherein the composition concentrate comprises 5-15% (v/v) of water, e.g. 6-15% (v/v) or 7-13% (v/v), such as about 10% (v/v).
11. The storage stable liquid composition concentrate according to any one of clauses 1 to 10, wherein the viscosity of the composition concentrate at 21° C. is below 90 mPa·s, suitably as determined using the Viscosity Assessment Method described herein using capillary extrusion rheometer (mVROC instrument).
12. The storage stable liquid composition concentrate according to any one of clauses 1 to 11, wherein the composition concentrate further comprises a salt.
13. The storage stable liquid composition concentrate according to clause 12, wherein the salt is present at a concentration of 1-50 mM.
14. The storage stable liquid composition concentrate according to clause 12 or clause 13, wherein the salt is sodium chloride, and is suitably present at a concentration of 20-40 mM, such as about 25 mM.
15. The storage stable liquid composition concentrate according to clause 12 or clause 13, wherein the salt is calcium chloride, and is suitably present at a concentration of 5-10 mM, such as about 5 mM.
16. The storage stable liquid composition concentrate according to any one of clauses 1 to 15, wherein the composition concentrate further comprises a sugar.
17. The storage stable liquid composition concentrate according to clause 16, wherein the sugar is selected from the group consisting of glucose, sucrose, trehalose, raffinose, stachyose and verbascose, and is suitably sucrose.
18. The storage stable liquid composition concentrate according to clause 16 or clause 17, wherein the sugar is present at a concentration of 1-50 mM, e.g. 20-40 mM, such as about 25 mM.
19. The storage stable liquid composition concentrate according to any one clauses 1 to
18, further comprising an antioxidant.
20. The storage stable liquid composition concentrate according to clause 19, wherein the antioxidant is selected from the group consisting of monothioglycerol, butylated hydroxyanisole, glutathione (reduced), and methionine, and is suitably monothioglycerol.
21. The storage stable liquid composition concentrate according to clause 19 or clause 20, wherein the antioxidant is present at a concentration of 1-10 mg/mL, such as 1-5 mg/mL e.g. 5 mg/mL.
22. The storage stable liquid composition concentrate according to any one of clauses 1 to 21, wherein the pH of the composition concentrate is in the range 2.5-4.0, such as in the range 2.5-3.5, or is about 3.0.
23. The storage stable liquid composition concentrate according to any one of clauses 1 to 22, further comprising a buffer which is suitably selected from the group consisting of citrate, formate, glycine, alanine, aspartate, malate, glyoxylate, gluconate, lactate, glycolate, tartrate and succinate and especially comprising lactate as buffer.
24. The storage stable liquid composition concentrate according to any one of clauses 1 to 23, wherein the composition concentrate is substantially free of, or free of organic small molecule solvents of molecular weight 50-120 Da.
25. The storage stable liquid composition concentrate according to any one of clauses 1 to 24, wherein the composition concentrate is substantially free of, or free of propylene glycol.
26. The storage stable liquid composition concentrate according to any one of clauses 1 to 25, wherein the composition concentrate is substantially free of, or free of N,N-dimethylacetamide, dimethyl sulfoxide, 1-methyl-2-pyrrolidinone, ethanol, isopropyl alcohol and benzyl alcohol.
27. The storage stable liquid composition concentrate according to any one of clauses 1 to 26, wherein the composition concentrate is substantially free of, or free of cyclodextrin.
28. The storage stable liquid composition concentrate according to any one of clauses 1 to 27, further comprising a non-ionic surfactant.
29. The storage stable liquid composition concentrate according to clause 28, wherein the non-ionic surfactant is selected from the group consisting of an alkyl glycoside, a polysorbate, an alkyl ether of polyethylene glycol, a block copolymer of polyethylene glycol and polypropylene glycol, and an alkylphenyl ether of polyethylene glycol.
30. The storage stable liquid composition concentrate according to clause 29, wherein the non-ionic surfactant is an alkyl glycoside which is suitably selected from the group consisting of dodecyl maltoside, dodecyl glucoside, octyl glucoside, octyl maltoside, decyl glucoside, decyl maltoside, decyl glucopyranoside, tridecyl glucoside, tridecyl maltoside, tetradecyl glucoside, tetradecyl maltoside, hexadecyl glucoside, hexadecyl maltoside, sucrose monooctanoate, sucrose monodecanoate, sucrose monododecanoate, sucrose monotridecanoate, sucrose monotetradecanoate and sucrose monohexadecanoate; and in particular is dodecyl maltoside or decyl glucopyranoside, especially dodecyl maltoside.
31. The storage stable liquid composition concentrate according to clause 29, wherein the non-ionic surfactant is a polysorbate which is suitably polysorbate 20 or polysorbate 80.
32. The storage stable liquid composition concentrate according to clause 29, wherein the non-ionic surfactant is an alkyl ether of polyethylene glycol which is suitably selected from the group consisting of polyethylene glycol (2) hexadecyl ether (Brij 52), polyethylene glycol (2) oleyl ether (Brij 93), polyethylene glycol (2) dodecyl ether (Brij L4), polyethylene glycol (4) lauryl ether (Brij 30), polyethylene glycol (10) lauryl ether (Brij 35), polyethylene glycol (20) hexadecyl ether (Brij 58) and polyethylene glycol (10) stearyl ether (Brij 78).
33. The storage stable liquid composition concentrate according to clause 29, wherein the non-ionic surfactant is a block copolymer of polyethylene glycol and polypropylene glycol, which is suitably selected from the group consisting of poloxamer 188, poloxamer 407, poloxamer 171 or poloxamer 185.
34. The storage stable liquid composition concentrate according to clause 29, wherein the non-ionic surfactant is alkylphenyl ether of polyethylene glycol, which is suitably 4-(1,1,3,3-tetramethylbutyl)phenyl-polyethylene glycol.
35. The storage stable liquid composition concentrate according to any one of clauses 29 to 34, wherein the non-ionic surfactant is present at a concentration of 1-5000 μg/mL, 1-1000 μg/mL, such as 5-500 μg/mL, 10-400 μg/mL, 20-400 μg/mL, 50-400 μg/mL, 10-300 μg/mL, 20-300 μg/mL, 50-300 μg/mL, 10-200 μg/mL, 20-200 μg/mL, 50-200 μg/mL, 10-100 μg/mL, 20-100 μg/mL, 50-100 μg/mL or around 50 μg/mL.
36. The storage stable liquid composition concentrate according to any one of clauses 1 to 35, further comprising a preservative such as a phenolic or benzylic preservative.
37. The storage stable liquid composition concentrate according to clause 36, wherein the phenolic or benzylic preservative is selected from the group consisting of phenol, m-cresol, chlorocresol, benzyl alcohol, propyl paraben and methyl paraben.
38. The storage stable liquid composition concentrate according to clause 1, comprising bendamustine or a pharmaceutically acceptable salt thereof and a liquid solvent in which said bendamustine or a pharmaceutically acceptable salt thereof is dissolved; wherein said composition consists of or consists essentially of:
   (i) bendamustine or a pharmaceutically acceptable salt thereof (e.g. at a concentration of 45-75 mM, such as about 61 mM);
   (ii) a mixture of two different polyethylene glycol (PEG) polymers selected from the group consisting of PEG200, PEG300, PEG400 and PEG600; wherein said mixture is present in a total concentration of 80-95% (v/v) and
   (iii) 5-20% (v/v) of water;
   (iv) optionally an inorganic salt containing a metal cation, especially a Group 1 or 2 metal cation;
   (v) optionally a sugar;
   (vi) optionally an antioxidant
   (vii) optionally a non-ionic surfactant;
   (viii) optionally a preservative; and
   (ix) optionally a buffer;
   wherein the pH of the composition concentrate is in the range 2.5-4.0 (e.g. in the range 2.5-3.5).
39. A storage stable liquid composition concentrate comprising bendamustine or a pharmaceutically acceptable salt thereof and a liquid solvent in which said bendamustine or a pharmaceutically acceptable salt thereof is dissolved;
   wherein said composition comprises:
   (i) a mixture of two or more different polyethylene glycol (PEG) polymers,
   wherein each polyethylene glycol has an average molecular weight in the range 150-650 Da; and
   (ii) 5-20% (v/v) of water;
wherein the composition is substantially free of, or free of ethanol and is substantially free of, or free of cyclodextrin.
40. A storage stable liquid composition concentrate according to clause 39 wherein at least one of the different polyethylene glycol (PEG) polymers is PEG300.
41. The storage stable liquid composition concentrate according to any one of clauses 1 to 40, which is a pharmaceutical composition.
42. The storage stable liquid composition concentrate according to any one of clauses 1 to 41, which is a composition concentrate for use in therapy.
43. The storage stable liquid composition concentrate according to any one of clauses 1 to 40, or pharmaceutical composition according to clause 41, for use in treating chronic lymphocytic leukaemia (CLL), multiple myeloma or non-Hodgkin lymphoma (NHL).
44. A method of treating chronic lymphocytic leukaemia (CLL), multiple myeloma or non-Hodgkin lymphoma (NHL) which comprises administering to a patient in need thereof a therapeutically effective amount of a diluted form (ready-to-administer) of a storage stable liquid composition concentrate according to any one of clauses 1 to 40, or a pharmaceutical composition according to clause 39.
45. The storage stable liquid composition concentrate or pharmaceutical composition for use according to clause 43 or the method according to clause 44, for use in combination with a further therapeutic agent.
46. The storage stable liquid composition concentrate for use, pharmaceutical composition for use or the method according to clause 45, wherein the further therapeutic agent is selected from the group consisting of etoposide, rituximab, fludarabine, mitoxantrone, methotrexate, prednisone, vincristine and ⁹⁰Y-ibritumomab tiuxetan.
47. The storage stable liquid composition concentrate for use, pharmaceutical composition for use or the method according to any one of clauses 43 to 45, wherein the composition concentrate is diluted prior to administration.
48. The storage stable liquid composition concentrate for use, pharmaceutical composition for use or the method according to clause 47, wherein the composition concentrate is diluted with a diluent e.g. selected from isotonic saline (0.9% w/v), isotonic dextrose (5% w/v), isotonic mixtures of saline and dextrose (e.g. saline (0.45% w/v) and dextrose (2.5% w/v)), sterile water for injection and bacteriostatic water for injection.
49. The storage stable liquid composition concentrate for use, pharmaceutical composition for use or the method according to clause 47 or clause 48, wherein the concentration of bendamustine or pharmaceutically acceptable salt thereof in the diluted composition (ready-to-administer) is 7-25% of the concentration in the composition concentrate.
50. The storage stable liquid composition concentrate for use, pharmaceutical composition for use or the method according to any one of clauses 47 to 49, wherein the concentration of bendamustine or a pharmaceutically acceptable salt thereof in the diluted composition (ready-to-administer) is 1-60 mM, such as 1-50 mM, 1-25 mM, 2-25 mM, 3-20 mM, 4-15 mM or 4.5-13.6 mM.
51. The storage stable liquid composition concentrate for use, pharmaceutical composition for use, or the method according to any one of clauses 47 to 50, wherein following dilution of the composition concentrate, the diluted composition (ready-to-administer) is administered via intravenous infusion.
52. A method of preparing a solution for intravenous administration (ready-to-administer) by diluting a composition concentrate according to any one of clauses 1 to 40, with a diluent e.g. selected from isotonic saline (0.9% w/v), isotonic dextrose (5% w/v), isotonic mixtures of saline and dextrose (e.g. saline (0.45% w/v) and dextrose (2.5% w/v)), sterile water for injection and bacteriostatic water for injection.
53. The method according to clause 52, wherein the concentration of bendamustine or a pharmaceutically acceptable salt thereof in the diluted composition (ready-to-administer) is 1-60 mM, such as 1-50 mM, 1-25 mM, 2-25 mM, 3-20 mM, 4-15 mM or 4.5-13.6 mM.
54. The method according to clause 52 or clause 53, wherein the concentration of bendamustine or pharmaceutically acceptable salt thereof in the diluted composition (ready-to-administer) is 7-25% of the concentration in the composition concentrate.
55. The method according to any one of clauses 52 to 54, wherein the concentration of bendamustine or a pharmaceutically acceptable salt thereof in the diluted composition (ready-to-administer) is 1-60 mM, such as 1-50 mM, 1-25 mM, 2-25 mM, 3-20 mM, 4-15 mM or 4.5-13.6 mM.
56. A ready-to-administer composition for intravenous administration of bendamustine or a pharmaceutically acceptable salt thereof, prepared according to the method of any one of clauses 52 to 55.
57. A ready-to-administer composition comprising bendamustine or a pharmaceutically acceptable salt thereof, prepared by diluting a composition concentrate according to any one of claims 1 to 40 with a diluent e.g. selected from isotonic saline (0.9% w/v), isotonic dextrose (5% w/v), isotonic mixtures of saline and dextrose (e.g. saline (0.45% w/v) and dextrose (2.5% w/v)), sterile water for injection and bacteriostatic water for injection.
58. The ready-to-administer composition according to clause 57, wherein the concentration of bendamustine or a pharmaceutically acceptable salt thereof in the diluted composition (ready-to-administer) is 1-60 mM, such as 1-50 mM, 1-25 mM, 2-25 mM, 3-20 mM, 4-15 mM or 4.5-13.6 mM.
59. The ready-to-administer composition to clause 57 or clause 58, wherein the concentration of bendamustine or pharmaceutically acceptable salt thereof is 7-25% e.g. 10% of the concentration in the composition concentrate.
60. The ready-to-administer composition according to any one of clauses 57 to 59, wherein the concentration of bendamustine or a pharmaceutically acceptable salt thereof is 1-60 mM, such as 1-50 mM, 1-25 mM, 2-25 mM, 3-20 mM, 4-15 mM or 4.5-13.6 mM.
61. A vial which is suitably a glass vial containing the storage stable liquid composition concentrate according to any one of clauses 1 to 40.
62. The vial according to clause 61, which is a multiple dose vial.

The composition concentrates of the invention are expected to have one or more of the following advantages: they are ready-to-dilute composition concentrates of bendamustine which have good storage stability at 2-8° C. and at higher temperatures, they contain excipients which are approved for intravenous (IV) use in humans, and they have low viscosity and therefore improved dosing accuracy and handling. Lower viscosity of the ready-to-dilute composition concentrate results in lower adherence of the composition to the internal walls of the storage container as well as the syringe barrel and syringe needle during the dose preparation steps. Lower adherence minimises losses during the dose preparation and thus improves dose accuracy. In addition, lower viscosity of the composition results in lower physical force required for drawing the composition from the storage container into the syringe and for dispensing the composition from the syringe into the IV bag prior to administration. The lower physical force required throughout the procedure improves the ease and convenience of handling which can further contribute to minimisation of dosing errors.

### EXAMPLES

### General Methods

### Reversed-Phase Chromatography (RP-HPLC)

High performance reverse phase chromatography is performed using the Thermo-Scientific Ultimate 3000 HPLC with Diode Array Detector (DAD3000) with a Inertsil ODS2 column, of 250 mm by 4.6 mm, 5 μm particle size and 150 Å pore size. Mobile Phase A is a mixture of water and trifluoroacetic acid in the ratio of 1,000:1 (v/v). Mobile Phase B is a mixture of acetonitrile and trifluoroacetic acid, in the ratio of 1,000:1 (v/v). The sample comprising bendamustine (or pharmaceutically acceptable salt thereof) is bound in Mobile Phase A and eluted using a gradient of Mobile Phase A and Mobile Phase B. The sample volume is 10 microlitres, the flow rate is 1.0 mL/min, with 254 nm UV detection. All analyses are performed at 27° C.

### Visual Assessment Method

Visible particles are suitably detected using the 2.9.20. European Pharmacopoeia Monograph (Particulate Contamination: Visible Particles). The apparatus required consists of a viewing station comprising:
a matt black panel of appropriate size held in a vertical position
a non-glare white panel of appropriate size held in a vertical position next to the black panel
an adjustable lampholder fitted with a suitable, shaded, white-light source and with a suitable light diffuser (a viewing illuminator containing two 13 W fluorescent tubes, each 525 mm in length, is suitable). The intensity of illumination at the viewing point is maintained between 2000 lux and 3750 lux. Any adherent labels are removed from the container and the outside washed and dried. The container is gently swirled or inverted, ensuring that air bubbles are not introduced, and observed for about 5 seconds in front of the white panel. The procedure is repeated in front of the black panel. The presence of any particles is recorded. The visual scores are ranked as follows:

### Visual Assessment Scoring Method A

Visual score 1: clear solution free of particles
Visual score 2: slight particle formation, detectable via the viewing station but not under normal light
Visual score 3: more significant precipitation, clearly visible under normal daylight
Visual scores 1 and 2 are considered pharmaceutically acceptable for injectable products; visual score 3 is not considered pharmaceutically acceptable.

### Viscosity Assessment Method

Viscosity is measured using capillary extrusion rheometer (mVROC manufactured by RheoSense). Shear rate is controlled by a positive displacement pump that injects the sample or formulation at a controlled rate, and pressure drop is measured as the formulation passes over sensors in the flow field. This data is used by the instrument software to calculate the viscosity of the sample using a derivation of the Hagen-Poiseuille equation. The instrument can either be set to a controlled flow rate or a defined shear rate for performing the measurement. Set controlled flow rate was used in the experiments described herein. Reduction of the flow rate for the more viscous test formulations was required, compared to water, to ensure the signal was within the acceptable range for software calculations.

Water is used as a control; 1 mL is loaded into the sample syringe, that is mounted in the instrument, and measured before sample testing. It is run at a fixed flow of 25 μl/min (that corresponds to a shear rate of 178 s⁻¹) initially for 60 seconds to prime the chip and then a further 3 minutes for measurement, with a 30 second delay before collecting data. The viscosity of water must be determined by the instrument as 1 mPa·s to show it is performing correctly.

The sample is equilibrated to room temperature, and the instrument set to 21° C. 1 mL sample is loaded and it is run at 16.3 μl/min for 60 seconds to prime and for a further 3 minutes for measurement, with a 30 second delay before collecting data.

### Example 1: Composition Examples

The following example compositions may be prepared. It should be noted that if a % (v/v) is not specified for a liquid composition component, then the volume is considered to be negligible compared to the overall volume of the composition.

### Example A

| Bendamustine hydrochloride | 25 mg/mL |
|---|---|
| PEG300 | 79% (v/v) |
| PEG400 | 11% (v/v) |
| water for injection (WFI) | 10% (v/v) |
| monothioglycerol | 5 mg/mL |
| pH | 3.0 |

### Example B

| Bendamustine hydrochloride | 25 mg/mL |
|---|---|
| PEG300 | 79% (v/v) |
| PEG400 | 11% (v/v) |
| WFI | 10% (v/v) |
| sodium chloride | 25 mM |
| monothioglycerol | 5 mg/mL |
| pH | 3.0 |

### Example C

| Bendamustine hydrochloride | 25 mg/mL |
|---|---|
| PEG300 | 79% (v/v) |
| PEG400 | 11% (v/v) |
| WFI | 10% (v/v) |
| sucrose | 25 mM |
| monothioglycerol | 5 mg/mL |
| pH | 3.0 |

### Example D

| Bendamustine hydrochloride | 25 mg/mL |
|---|---|
| PEG300 | 68% (v/v) |
| PEG400 | 22% (v/v) |
| WFI | 10% (v/v) |
| sodium chloride | 25 mM |
| monothioglycerol | 5 mg/mL |
| pH | 3.0 |

### Example E

| Bendamustine hydrochloride | 25 mg/mL |
|---|---|
| PEG300 | 45% (v/v) |
| PEG400 | 45% (v/v) |
| WFI | 10% (v/v) |
| sodium chloride | 25 mM |
| monothioglycerol | 5 mg/mL |
| pH | 3.0 |

### Example F

| Bendamustine hydrochloride | 25 mg/mL |
|---|---|
| PEG300 | 22% (v/v) |
| PEG400 | 68% (v/v) |
| WFI | 10% (v/v) |
| sodium chloride | 25 mM |
| monothioglycerol | 5 mg/mL |
| pH | 3.0 |

### Example G

| Bendamustine hydrochloride | 25 mg/mL |
|---|---|
| PEG300 | 81% (v/v) |
| PEG400 | 14% (v/v) |
| WFI | 5% (v/v) |
| sodium chloride | 25 mM |
| monothioglycerol | 5 mg/mL |
| pH | 3.0 |

### Example H

| Bendamustine hydrochloride | 25 mg/mL |
|---|---|
| PEG300 | 70% (v/v) |
| PEG400 | 10% (v/v) |
| WFI | 20% (v/v) |
| sodium chloride | 25 mM |
| monothioglycerol | 5 mg/mL |
| pH | 3.0 |

### Example 2: Method for Preparation of Bendamustine Compositions

Bendamustine compositions described in subsequent examples are prepared using the following steps:
Solvent for the composition is prepared by mixing the organic part(s) (i.e. selected PEG(s) and/or propylene glycol) with water in the % volume ratios (v/v) stated in example Tables 2, 3, 4, 5, 6, and 7. For example, for a composition comprising
79% (v/v) of PEG300, 11% (v/v) PEG400 and 10% (v/v) water, 79 volume parts of PEG300 are mixed with 11 volume parts of PEG400 and 10 volume parts of water.
All other components of the composition, such as salts, sugars, antioxidants or buffers are added to the water part of the composition prior to being mixed with the organic part(s). The additional components are used at very low concentration and their impact on the volume ratios of the individual solvents of the composition is negligible.
The solvent mixture comprising other dissolved components is then thoroughly mixed by stirring with magnetic stir bar at room temperature for at least 30 minutes. pH is adjusted to the desired level by addition of concentrated sodium hydroxide e.g. 1M NaOH.
Bendamustine hydrochloride is added to the solvent mixture to achieve 25 mg/mL based on the amount of bendamustine hydrochloride. For example, for 10 mL of solvent-excipient mixture, 250 mg of bendamustine hydrochloride is added and the resultant mixture is stirred with magnetic stir bar at room temperature for at least 30 minutes. Water, where referred to in in the examples, is WFI. A final adjustment of the pH (if required) is made by addition of concentrated sodium hydroxide.e.g. 1M NaOH or concentrated hydrochloric acid e.g. 1M HCl.

### Example 3: Effect of pH on Stability of Bendamustine

The effect of pH on the stability of bendamustine compositions comprising 90% (v/v) PEG400 and 10% (v/v) water was investigated using the RP-HPLC and Visual Assessment methods described in General Methods. All compositions comprised 5 mg/mL monothioglycerol and 25 mg/mL bendamustine (as hydrochloride) and were prepared as stated in Example 2. All samples were clear and colourless solutions at the start of the experiment (T0). Tables 1 shows visual appearance of the compositions following storage at 30° C. and 2-8° C. Table 2 shows the increase in total impurities between TO and specified timepoints following storage 30° C. and 2-8° C.

TABLE 1
Stability of bendamustine compositions
assessed using Visual Assessment following storage
at 30° C. for 4 weeks and at 2-8° C. for 26 weeks

| | | Visual appearance | |
|---|---|---|---|
| | | 30° C. | 2-8° C. |
| Composition | pH | (4 weeks) | (26 weeks) |
| 1 | 3.0 | 1 | 1 |
| 2 | 3.6 | 1 | 1 |
| 3 | 4.0 | 1 | 1 |
| 4 | 4.5 | 2 | 3 |
| 5 | 5.15 | 3 | 3 |
| 6 | 9.25 | 3 | 3 |

TABLE 2
Stability of bendamustine compositions
assessed using RP-HPLC following storage at
30° C. for 1 month and at 2-8° C. for 6 months

| | | Δ Total Impurities (%) | |
|---|---|---|---|
| | | 30 ° C. | 2-8 ° C. |
| Composition | pH | (4 weeks) | (26 weeks) |
| 1 | 3.0 | 2.5 | 1.4 |
| 2 | 3.6 | 2.8 | 2.9 |
| 3 | 4.0 | 4.9 | 1.2 |
| 4 | 4.5 | 5.0 | ND* |
| 5 | 5.15 | ND* | ND* |
| 6 | 9.25 | ND* | ND* |

*Not determined-Samples could not be analysed due to precipitation

The results in Tables 1 and 2 show that the final pH of bendamustine compositions has a clear impact on the physical and chemical stability of bendamustine. As shown in Table 1, as the pH is increased above 4 there was a considerably greater propensity to precipitation. As shown in Table 2, increasing the pH also decreases the chemical stability of the bendamustine compositions, as shown by the increase in impurities and precipitation at higher pH values. Thus, the optimal pH is expected to be in the range 2.5 to 4.0, in particular between 2.5 and 3.5 e.g. between 2.5 and 3.0, and especially at around 3.0. All compositions in subsequent examples were prepared at about pH 3.0.

### Example 4: Stability of Bendamustine Compositions in the Presence of Different Types of PEG and Mixtures of PEG

The effect of various PEG compounds on the stability of bendamustine compositions was investigated using the RP-HPLC method described in General Methods. All compositions were prepared as stated in Example 2 and comprised 5 mg/mL monothioglycerol and 25 mg/mL bendamustine (as hydrochloride). All samples were at pH 3.0, clear and colourless solutions at the start of the experiment (T0).

Table 3 shows the change in % total impurities following storage at 30° C. for 4 weeks.

TABLE 3
Stability of bendamustine compositions assessed using
RP-HPLC following storage at 30° C. for 4 weeks

| | | | | | | Increase in total |
|---|---|---|---|---|---|---|
| | Propylene | PEG | PEG | PEG | | Impurities 30° C. |
| Composi- | glycol | 200 | 300 | 400 | Water | (4 weeks) |
| tion | % (v/v) | (%) | (%) | (%) | (%) | (%) |
| 7* | 10 | — | — | 90 | — | 1 |
| 8 | 9 | — | — | 81 | 10 | 2.5 |
| 9 | — | — | 79 | 11 | 10 | 1.34 |
| 10 | — | — | 68 | 22 | 10 | 1.56 |
| 11 | — | 95 | 0 | 0 | 5 | 2.28 |
| 12 | — | 0 | 95 | 0 | 5 | 3.38 |
| 13 | — | 0 | 0 | 95 | 5 | 3.55 |
| 14 | — | 47.5 | 0 | 47.5 | 5 | 0.86 |
| 15 | — | 90 | 0 | 0 | 10 | 2.4 |
| 16 | — | 0 | 90 | 0 | 10 | 4.1 |
| 17 | — | 0 | 0 | 90 | 10 | 3.5 |
| 18 | — | 45 | 0 | 45 | 10 | 1.9 |
| 19 | — | 30 | 60 | 0 | 10 | 1.7 |
| 20 | — | 20 | 60 | | 20 | 5.37 |
| 21 | — | 10 | 70 | | 20 | 4.05 |
| 22 | — | 20 | | 60 | 20 | 3.11 |
| 23 | — | 10 | | 70 | 20 | 3.47 |
| 24 | — | — | 20 | 60 | 20 | 3.23 |
| 25 | — | — | 10 | 70 | 20 | 5.37 |
| 26 | — | — | 60 | 20 | 20 | 2.89 |
| 27 | — | — | 70 | 10 | 20 | 2.77 |

*(Bendeka formulation)

Composition 7 corresponds to the marketed composition of Bendeka® which is non-aqueous and contains a mixture of propylene glycol and PEG400 as solvent. Comparing Compositions 7 and 8, it can be seen that adding a proportion of water (10% v/v, with a corresponding reduction in volume of propylene glycol/PEG400) results in a decrease in stability. However, as shown for Compositions 9 and 10, if the propylene glycol/PEG400 mixture is replaced with a mixture of PEG300/PEG400, the destabilizing effect of adding water to the composition is reduced.

Comparing Composition 7 (Bendeka®) with Compositions 11-13, it can be seen that adding 5% v/v of water and replacing the propylene glycol/PEG400 mixture with a single PEG compound results in a decrease in stability. However, if a mixture PEG compounds is used as in Composition 14 (PEG200/PEG400), the stability of the composition is actually improved compared to Bendeka®.

Mixtures of PEG200/PEG300 as well as PEG300/PEG400 and PEG200/PEG400 also demonstrate this unexpected improvement in stability compared with the mixture of propylene glycol/PEG400 and the use of a PEG alone, as can be seen by comparing compositions 15-19.

Compositions 20-27 have a higher proportion of water (20% v/v) and mixtures of different PEG compounds in different proportions. It can be seen that of these compositions, Compositions 26 and 27 with a mixture of PEG300/PEG400 are most stable, with an excess of PEG300 appearing to be optimal.

### Example 5: Effect of Water Concentration on Stability of Bendamustine Compositions

The effect of concentration of water on the stability of bendamustine compositions comprising a fixed ratio of PEG200 and PEG300 was investigated using the RP-HPLC method described in General Methods. All compositions were prepared as stated in Example 2 and comprised 5 mg/mL monothioglycerol and 25 mg/mL bendamustine (as hydrochloride). All samples were at pH 3.0, clear and colourless solutions at the start of the experiment (T0). Table 4 shows the change in % total impurities following storage at 30° C. for 4 weeks.

TABLE 4
Stability of bendamustine compositions assessed using
RP-HPLC following storage at 30° C. for 4 weeks

| | | | | Increase in |
|---|---|---|---|---|
| | | | | total Impurities |
| | | | | 30° C. |
| | PEG200 | PEG300 | Water | (4 weeks) |
| Composition | (%) | (%) | (%) | (%) |
| 28 | 33 | 66 | 0 | 1.21 |
| 29 | 30 | 60 | 10 | 2.06 |
| 30 | 27 | 53 | 20 | 6.04 |
| 31 | 24 | 46 | 30 | 9.49 |
| 32 | 20 | 40 | 40 | 17.77 |

The results in Table 4 show that the greater the concentration of water in the bendamustine composition, the greater the increase in impurities following storage.

### Example 6: Viscosity of Bendamustine Compositions

The viscosity of the composition used in the marketed formulation of bendamustine (Bendeka®) was compared with that of compositions based on mixtures of PEGs and 10% water using the Viscosity Assessment Method described in General Methods. All formulations were prepared as for Example 4, with the exception that for viscosity testing the formulations were tested in the absence of bendamustine hydrochloride due to safety reasons. All samples were at pH 3.0, clear and colourless solutions at the start of the experiment (T0). The results are shown in Table 5.

TABLE 5
Viscosity of bendamustine compositions

| | Propylene glycol | PEG200 | PEG300 | PEG400 | Water | Viscosity |
|---|---|---|---|---|---|---|
| Composition | (%) | (%) | (%) | (%) | (%) | (mPa · s) |
| 7 (Bendeka | 10 | 0 | 0 | 90 | 0 | 94.2 |
| formulation) | | | | | | |
| 9 | 0 | 0 | 79 | 11 | 10 | 62.1 |
| 10 | 0 | 0 | 68 | 22 | 10 | 67.7 |

It can be seen from Table 5 that compositions comprising mixtures of PEG300 and PEG400 in the presence of 10% (v/v) water have a considerably lower viscosity than the composition of the commercial product, Bendeka®.

### Example 7: The Effect on Sodium Chloride on the Stability of Bendamustine Compositions in the Presence of a Mixtures of PEGs

The effect of sodium chloride on the stability of bendamustine compositions containing a mixture of PEGs was investigated using the RP-HPLC method described in General Methods. Compositions were prepared as stated in Example 2 and comprised 5 mg/mL monothioglycerol and 25 mg/mL bendamustine (as hydrochloride). All samples were at pH 3.0, clear and colourless solutions at the start of the experiment (T0). Table 6 shows the change in % total impurities following storage at 30° C. for 4 weeks.

TABLE 6
Stability of bendamustine compositions assessed using
RP-HPLC following storage at 30° C. for 4 weeks

| | | | | | Increase |
|---|---|---|---|---|---|
| | | | | | in total |
| | | | | | impurities |
| | Sodium | | | | 30° C. |
| | Chloride | PEG300 | PEG400 | Water | (4 weeks) |
| Composition | mM | (%) | (%) | (%) | (%) |
| 9 | 0 | 79 | 11 | 10 | 1.3 |
| 33 | 5 | 79 | 11 | 10 | 1.1 |
| 34 | 25 | 79 | 11 | 10 | 1.1 |
| 35 | 50 | 79 | 11 | 10 | 1.2 |

The results in Table 6 show that the addition of sodium chloride (particularly at 5 and 25 mM) resulted in a slight improvement in bendamustine stability.

### Example 8: The Effect of Sucrose on the Stability of Bendamustine Compositions in the Presence of a Mixtures of PEGs

The effect of sucrose on the stability of bendamustine compositions containing a mixture of PEGs was investigated using the RP-HPLC method described in General Methods. Compositions were prepared as stated in Example 2 and comprised 5 mg/mL monothioglycerol and 25 mg/mL bendamustine (as hydrochloride). All samples were at pH 3.0, clear and colourless solutions at the start of the experiment (T0). Table 7 shows the change in % total impurities following storage at 30° C. for 4 weeks.

TABLE 7
Stability of bendamustine compositions assessed using
RP-HPLC following storage at 30° C. for 4 weeks

| | | | | | Δ Total |
|---|---|---|---|---|---|
| | | | | | impurities |
| | | | | | 30° C. |
| | Sucrose | PEG 300 | PEG 400 | Water | (4 weeks) |
| Composition | mM | (%) | (%) | (%) | (%) |
| 9 | 0 | 79 | 11 | 10 | 1.3 |
| 36 | 5 | 79 | 11 | 10 | 1.2 |
| 37 | 25 | 79 | 11 | 10 | 1.1 |
| 38 | 50 | 79 | 11 | 10 | 1.4 |

The results in Table 7 show that the addition of sucrose (particularly at 5 and 25 mM) resulted in a slight improvement in bendamustine stability.

### Example 9: Long-Term Stability of Bendamustine in Compositions Comprising a Mixture of PEGs and an Antioxidant or a Buffer

Stability of bendamustine in compositions comprising a mixture of PEG300 and PEG400 was assessed at 2-8° C. for 6 months using the RP-HPLC method described in General Methods. Compositions were prepared as stated in Example 2. The compositions comprised 25 mg/ml bendamustine (as hydrochloride) and additional components shown in Table 8 and were adjusted to pH 3.0. In the method of Example 2 to prepare composition 40, lactate was added to the composition in the form of lactic acid with adjustment of the pH to pH 3.0 using concentrated sodium hydroxide as stated.

Table 9 shows the change in % total impurities following storage at 2-8° C. for 6 months. The data show very good stability of bendamustine during storage at 2-8° C. Both compositions 39 and 40 which respectively contained the antioxidant monothioglycerol and the buffer lactate remained clear and colourless up to the 6 months time-point.

TABLE 8
Compositions of bendamustine tested for stability at 2-8° C.

| | Sodium | | | Monothioglycerol | Lactate | |
|---|---|---|---|---|---|---|
| | Chloride | PEG300 | PEG400 | (anti-oxidant) | (buffer) | Water |
| Composition | mM | (%) | (%) | mg/mL | mM | (%) |
| 39 | 25 | 79 | 11 | 5 | 0 | 10 |
| 40 | 0 | 79 | 11 | 0 | 46.5 | 10 |

TABLE 9
Stability of bendamustine compositions assessed using
RP-HPLC following storage at 2-8° C. 6 months

| | % Total impurities (RP-HPLC) | | | | |
|---|---|---|---|---|---|
| Composition | 0 months | 1 month | 2 months | 3 months | 6 months |
| 39 | 0.15 | 0.74 | 0.81 | 0.82 | 1.20 |
| 40 | 0.14 | 0.40 | 0.74 | 0.90 | 1.75 |

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

All patents, patent applications and references mentioned throughout the specification of the present invention are herein incorporated in their entirety by reference.

The invention embraces all combinations of preferred and more preferred groups and suitable and more suitable groups and embodiments of groups recited above.

1. A storage stable liquid composition concentrate comprising bendamustine or a pharmaceutically acceptable salt thereof and a liquid solvent in which said bendamustine or a pharmaceutically acceptable salt thereof is dissolved;
wherein said composition comprises:
i) a mixture of two or more different polyethylene glycol (PEG) polymers, wherein each polyethylene glycol has an average molecular weight in the range 150-650 Da; and
(ii) 5-20% (v/v) of water.

2. The storage stable liquid composition concentrate according to claim 1,
wherein the bendamustine form is bendamustine hydrochloride; and/or
wherein the concentration of bendamustine or pharmaceutically acceptable salt thereof is 25-250 mM, e.g. 25-225 mM, 25-200 mM, 25-175 mM, 25-150 mM, 25-125 mM, 25-100 mM, 25-75 mM, 35-250 mM, 35-225 mM, 35-200 mM, 35-175 mM, 35-150 mM, 35-125 mM, 35-100 mM, 35-75 mM, 45-250 mM, 45-225 mM, 45-200 mM, 45-175 mM, 45-150 mM, 45-125 mM, 45-100 mM, or 45-75 mM, such as about 61 mM.

3. The storage stable liquid composition concentrate according to claim 1, wherein the composition concentrate comprises two different polyethylene glycol (PEG) polymers, wherein each polyethylene glycol has an average molecular weight in the range 150-650 Da.

4. The storage stable liquid composition concentrate according to claim 3, wherein the two different polyethylene glycol (PEG) polymers are selected from the group consisting of PEG200, PEG300, PEG400 and PEG600.

5. The storage stable liquid composition concentrate according to claim 3, wherein in the mixture of two different polyethylene glycol (PEG) polymers, the polyethylene glycol polymer with lower average molecular weight is present in an equal or a greater proportion (especially a greater proportion) than the other polyethylene glycol polymer (% v/v).

6. The storage stable liquid composition concentrate according to claim 3, wherein the ratio of lower average molecular weight PEG to higher average molecular weight PEG is between 1.5:1 and 10:1 (% v/v), between 3:1 and 8:1 (% v/v), or about 7:1 (% v/v).

7. The storage stable liquid composition concentrate according to claim 3, wherein the mixture of two different polyethylene glycol (PEG) polymers is present in a total amount of 80-95% (v/v).

8. The storage stable liquid composition concentrate according to claim 1, wherein the composition comprises 5-15% (v/v), 6-15% (v/v), 7-13% (v/v), or about 10% (v/v) of water.

9. The storage stable liquid composition concentrate according to claim 1, wherein the viscosity of the composition concentrate at 21° C. is below 90 mPa·s, suitably as determined using the Viscosity Assessment Method described herein using capillary extrusion rheometer (mVROC instrument).

10. The storage stable liquid composition concentrate according to claim 1, further comprising sodium chloride at a concentration of 20-40 mM; or calcium chloride at a concentration of 5-10 mM; and/or
further comprising a sugar selected from the group consisting of glucose, sucrose, trehalose, raffinose, stachyose and verbascose; and/or
further comprising an antioxidant selected from the group consisting of monothioglycerol, butylated hydroxyanisole, glutathione (reduced) and methionine.

11.-12. (canceled)

13. The storage stable liquid composition concentrate according to claim 1, wherein the pH of the composition concentrate is in the range 2.5-4.0.

14. The storage stable liquid composition concentrate according to claim 1, wherein the composition concentrate is substantially free of, or free of ethanol.

15. The storage stable liquid composition concentrate according to claim 1 wherein the composition concentrate is substantially free of, or free of cyclodextrin.

16. (canceled)

17. The storage stable liquid composition concentrate according to claim 1, wherein said composition consists of or consists essentially of:
(i) bendamustine or a pharmaceutically acceptable salt thereof at a concentration of 45-75 mM;
(ii) a mixture of two different polyethylene glycol (PEG) polymers selected from the group consisting of PEG200, PEG300, PEG400 and PEG600; wherein said mixture is present in a total concentration of 80-95% (v/v) and
(iii) 5-20% (v/v) of water;
(iv) optionally an inorganic salt containing a metal cation;
(v) optionally a sugar;
(vi) optionally an antioxidant;
(vii) optionally a non-ionic surfactant;
(viii) optionally a preservative; and
(ix) optionally a buffer;
wherein the pH of the composition concentrate is in the range 2.5-4.0.

18.-19. (canceled)

20. A method of treating chronic lymphocytic leukaemia (CLL), multiple myeloma or non-Hodgkin lymphoma (NHL) which comprises administering to a patient in need thereof a therapeutically effective amount of a diluted form (ready-to-administer) of a storage stable liquid composition concentrate according to claim 1.

21. The method according to claim 20,
wherein the composition concentrate is diluted with a diluent e.g. selected from isotonic saline (0.9% w/v), isotonic dextrose (5% w/v), sotonic mixtures of saline and dextrose (e.g. saline (0.45% w/v) and dextrose (2.5% w/v)), sterile water for injection and bacteriostatic water for injection;
and/or wherein the concentration of bendamustine or pharmaceutically acceptable salt thereof in the diluted composition (ready-to-administer) is 7-25% of the concentration in the composition concentrate; and/or
wherein the concentration of bendamustine or a pharmaceutically acceptable salt thereof in the diluted composition (ready-to-administer) 4.5-13.6 mM.

22. The method according to claim 20, wherein following dilution of the composition, the diluted composition is administered via intravenous infusion.

23. A method of preparing a composition for intravenous administration by diluting a composition concentrate according to claim 1 with a diluent e.g. selected from isotonic saline (0.9% w/v), isotonic dextrose (5% w/v), isotonic mixtures of saline and dextrose (e.g. saline (0.45% w/v) and dextrose (2.5% w/v)), sterile water for injection and bacteriostatic water for injection.

24. (canceled)

25. A ready-to-administer composition comprising bendamustine or a pharmaceutically acceptable salt thereof, prepared by diluting a composition concentrate according to claim 1 with a diluent e.g. selected from isotonic saline (0.9% w/v), isotonic dextrose (5% w/v), isotonic mixtures of saline and dextrose (e.g. saline (0.45% w/v) and dextrose (2.5% w/v)), sterile water for injection and bacteriostatic water for injection.

26. A vial which is suitably a glass vial containing the storage stable liquid composition concentrate according to claim 1.

27. (canceled)
